(19) United States
(12) Patent Application Publication
(10) Pub. No.: US 2025/0215421 A1
(43) Pub. Date: Jul. 3, 2025

(54) ENRICHMENT OF CLONAL SUBSTRATES

(71) Applicant: LIFE TECHNOLOGIES CORPORATION, Carlsbad, CA (US)

(72) Inventor: Kylan Szeto, East Lyme, CT (US)

(21) Appl. No.: 18/990,488

(22) Filed: Dec. 20, 2024

Related U.S. Application Data
(60) Provisional application No. 63/616,010, filed on Dec. 29, 2023.

(51) Int. Cl. C12N 15/10

(52) U.S. Cl. CPC C12N 15/1065; C12N 15/1068

(57) ABSTRACT

A method for enriching clonal populations includes, for a population of target nucleic acids, includes exposing the population of target nucleic acids to a plurality of supports; amplifying the bound target nucleic acids in the presence of a primer to form supports including a plurality of copies of the target nucleic acids of the population of target nucleic acids; applying to the supports a capture primer; applying a magnetic bead functionalize with a moiety to bind to the binder moiety; and separating the first set of supports from the second and third set of supports.

### CROSS-REFERENCE TO RELATED APPLICATIONS

This application claims the benefit of U.S. Provisional Application No. 63/616,010, filed Dec. 29, 2023, which is incorporated herein by reference in its entirety.

### BACKGROUND

Increasingly, biological and medical research is turning to sequencing for enhancing biological studies and medicine. For example, biologist and zoologist are turning to sequencing to study the migration of animals, the evolution of species, and the origins of traits. The medical community is turned sequencing for studying the origins of disease, sensitivity to medicines, and the origins of infection. But sequencing has historically been an expensive process, thus limiting its practice.

Various sequencing techniques utilize supports having multiple copies of a target sequence. Often, there is an issue with making such supports, resulting in a blend of monoclonal supports and polyclonal supports. Generally, polyclonal supports reduce the number of usable reads when sequencing. As such, a method of reducing the number of polyclonal supports in a population of supports would be desirable.

### BRIEF DESCRIPTION OF THE DRAWINGS

The present disclosure may be better understood, and its numerous features and advantages made apparent to those skilled in the art by referencing the accompanying drawings.

FIG. 1 includes an illustration of an example sequencing system.

FIG. 2 includes an illustration of an example system including a sensor array.

FIG. 3 includes an illustration of an example sensor and associated well.

FIG. 4 includes an illustration of an example procedure for forming nucleic acid supports and loading such supports onto a substrate for sequencing.

FIG. 5 includes an illustration of another example procedure for forming nucleic acid supports and loading such supports onto a substrate for sequencing.

FIG. 6 includes an illustration of an example target including an enrichment section.

FIG. 7 includes an illustration of an example fusion primer including complements to the enrichment section.

FIG. 8 includes an illustration of a support and target nucleotide hybridized to a capture primer.

FIG. 9 includes an illustration of a support and target polynucleotide hybridized to a sequencing primer.

FIG. 10 includes an illustration of an example of an example method for isolating monoclonal supports.

FIG. 11 includes an illustration of an example method for applying the target polynucleotide to a support.

FIG. 12 includes an illustration of an example population of supports including monoclonal and polyclonal supports.

FIG. 13 includes an illustration of an example population of supports including monoclonal and polyclonal supports.

FIG. 14 includes an illustration of an example method for separating monoclonal supports from polyclonal supports.

FIG. 15 includes an illustration of an example method for separating monoclonal supports from polyclonal supports.

The use of the same reference symbols in different drawings indicates similar or identical items.

### DETAILED DESCRIPTION

In an example, target nucleic acids include an enrichment section including an enrichment barcode region and an enrichment primer region. The barcode sequence of the enrichment barcode region is selected from two or more sequences. Different target nucleic acids may include different barcode sequences in the enrichment barcode region. When amplified on a support, monoclonal supports have a population of target nucleic acids, each with an enrichment barcode region of the same barcode sequence-only one barcode sequence of the two or more barcode sequences-whereas polyclonal supports may have target nucleic acids, some having an enrichment barcode region having a first barcode sequence and some having an enrichment barcode region having a second barcode sequence different from the first. A method includes incubating a population of supports in the presence of a capture probe that includes a barcode sequence complementary to one of the two or more barcode sequences, an enrichment sequence complementary to the enrichment primer region, and a capture moiety. Magnetic beads including a moiety that binds to the capture moiety can be used to isolate those supports that have a target nucleic acid having an enrichment barcode region that is complementary to the barcode sequence of the capture probe. As such, monoclonal supports including target nucleic acids having an enrichment barcode region with a barcode sequence not complementary to the barcode sequence of the capture probe can be separated from polyclonal and monoclonal supports including target nucleic acids having an enrichment barcode region with a barcode sequence complementary to the barcode sequence of the capture probe. The process can be repeated using a different capture probe at least partially complementary to a different one of the two or more enrichment barcode sequences to capture different monoclonal support. Monoclonal supports recovered through the processes can be combined to provide a population of support with a higher concentration of monoclonal supports relative to polyclonal supports.

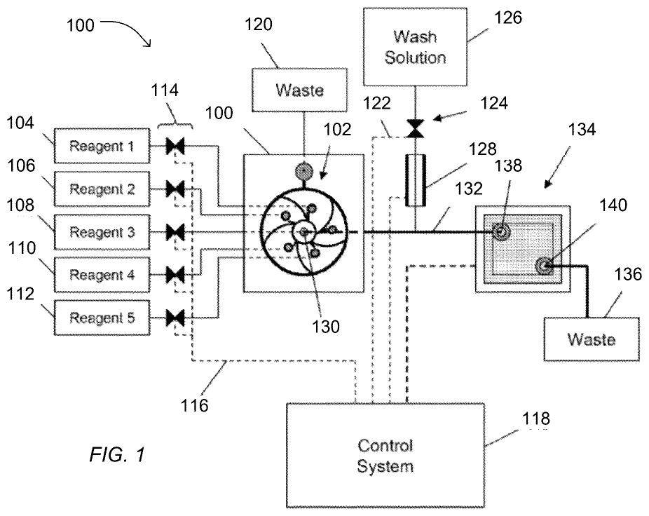

Such monoclonal supports can advantageously improve the performance of analysis techniques. For example, an increase in number of monoclonal supports relative to polyclonal supports can improve sequencing performance. For example, FIG. 1 diagrammatically illustrates a system for carrying out pH-based nucleic acid sequencing. Each electronic sensor of the apparatus generates an output signal that depends on the value of a reference voltage. The fluid circuit permits multiple reagents to be delivered to the reaction chambers.

In FIG. 1, a system 100 containing fluidics circuit 102, which is connected by inlets to at least two reagent reservoirs (104, 106, 108, 110, or 112), to waste reservoir 120, and to biosensor 134 by fluid pathway 132 that connects fluidics node 130 to inlet 138 of biosensor 134 for fluidic communication. Reagents from reservoirs (104, 106, 108, 110, or 112) can be driven to fluidic circuit 102 by a variety of methods including pressure, pumps, such as syringe pumps, gravity feed, and the like, and are selected by control of valves 114. Reagents from the fluidics circuit 102 can be driven through the valves 114 receiving signals from control system 118 to waste container 120. Reagents from the fluidics circuit 102 can also be driven through the biosensor 134 to the waste container 136. The control system 118 includes controllers for valves, which generate signals for opening and closing via electrical connection 116.

The control system 118 also includes controllers for other components of the system, such as wash solution valve 124 connected thereto by electrical connection 122, and reference electrode 128. Control system 118 can also include control and data acquisition functions for biosensor 134. In one mode of operation, fluidic circuit 102 delivers a sequence of selected reagents 1, 2, 3, 4, or 5 to biosensor 134 under programmed control of control system 118, such that in between selected reagent flows, fluidics circuit 102 is primed and washed, and biosensor 134 is washed. Fluids entering biosensor 134 exit through outlet 140 and are deposited in waste container 136 via control of pinch valve regulator. The valve is in fluidic communication with the sensor fluid output 140 of the biosensor 134.

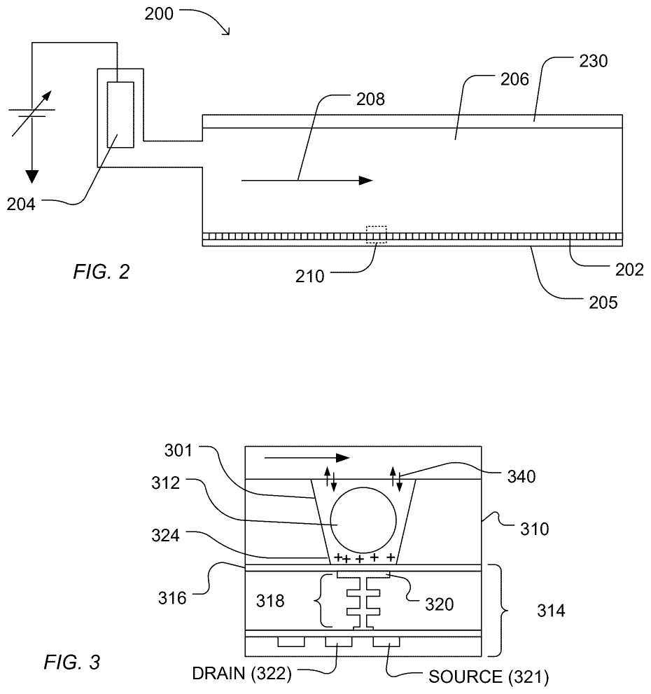

The device including the dielectric layer defining the well formed from the first access and second access and exposing a sensor pad finds particular use in detecting chemical reactions and byproducts, such as detecting the release of hydrogen ions in response to nucleotide incorporation, useful in genetic sequencing, among other applications. In a particular embodiment, a sequencing system includes a flow cell in which a sensory array is disposed, includes communication circuitry in electronic communication with the sensory array, and includes containers and fluid controls in fluidic communication with the flow cell. In an example, FIG. 2 illustrates an expanded and cross-sectional view of a flow cell 200 and illustrates a portion of a flow chamber 206. A reagent flow 208 flows across a surface of a well array 202, in which the reagent flow 208 flows over the open ends of wells of the well array 202. The well array 202 and a sensor array 205 together may form an integrated unit forming a lower wall (or floor) of flow cell 200. A reference electrode 204 may be fluidly coupled to flow chamber 206. Further, a flow cell cover 230 encapsulates flow chamber 206 to contain reagent flow 208 within a confined region.

FIG. 3 illustrates an expanded view of a well 301 and a sensor 314, as illustrated at 210 of FIG. 2. The volume, shape, aspect ratio (such as base width-to-well depth ratio), and other dimensional characteristics of the wells may be selected based on the nature of the reaction taking place, as well as the reagents, byproducts, or labeling techniques (if any) that are employed. The sensor 314 can be a chemical field-effect transistor (chemFET), more specifically an ion-sensitive FET (ISFET), with a floating gate 318 having a sensor plate 320 optionally separated from the well interior by a passivation layer 316. The sensor 314 can be responsive to (and generate an output signal related to) the amount of a charge 324 present on passivation layer 316 opposite the sensor plate 320. Changes in the charge 324 can cause changes in a current between a source 321 and a drain 322 of the chemFET. In turn, the chemFET can be used directly to provide a current-based output signal or indirectly with additional circuitry to provide a voltage-based output signal. Reactants, wash solutions, and other reagents may move in and out of the wells by a diffusion mechanism 340.

In an embodiment, reactions carried out in the well 301 can be analytical reactions to identify or determine characteristics or properties of an analyte of interest. Such reactions can generate directly or indirectly byproducts that affect the amount of charge adjacent to the sensor plate 320. If such byproducts are produced in small amounts or rapidly decay or react with other constituents, then multiple copies of the same analyte may be analyzed in the well 301 at the same time in order to increase the output signal generated. In an embodiment, multiple copies of an analyte may be attached to a solid phase support 312, either before or after deposition into the well 301. The solid phase support 312 may be microparticles, nanoparticles, beads, solid or porous comprising gels, or the like. For simplicity and ease of explanation, solid phase support 312 is also referred herein as a particle or bead. For a nucleic acid analyte, multiple, connected copies may be made by rolling circle amplification (RCA), exponential RCA, or like techniques, to produce an amplicon without the need of a solid support.

The well wall structure 310 can be formed of one or more layers of material. In an example, the well wall structure 310 can have a thickness (t) extending from the lower surface to the upper surface in a range of 0.3 micrometers to 10 micrometers, such as a range of 0.5 micrometers to 6 micrometers. The wells 301 can have a characteristic diameter, defined as the square root of 4 times the cross-sectional area (A) divided by Pi (e.g., sqrt (4*A/π), of not greater than 5 micrometers, such as not greater than 3.5 micrometers, not greater than 2.0 micrometers, not greater than 1.6 micrometers, not greater than 1.0 micrometers, not greater than 0.8 micrometers or even not greater than 0.6 micrometers. In an example, the characteristic diameter is at least 0.01 micrometers.

A bead support may be composed of organic polymers such as polystyrene, polyethylene, polypropylene, polyfluoroethylene, polyethyleneoxy, and polyacrylamide, as well as co-polymers and grafts thereof. A support may also be inorganic, such as glass, silica, controlled-pore-glass (CPG), or reverse-phase silica. The configuration of a support may be in the form of beads, spheres, particles, granules, a gel, or a surface. Supports may be porous or non-porous and may have swelling or non-swelling characteristics. In some embodiments, a support is an Ion Sphere Particle. Example bead supports are disclosed in U.S. Pat. No. 9,243,085, titled “Hydrophilic Polymeric Particles and Methods for Making and Using Same,” and in U.S. Pat. No. 9,868,826, titled “Polymer Substrates Formed from Carboxy Functional Acrylamide,” each of which is incorporated herein by reference.

In some embodiments, the solid support is a “microparticle,” “bead,” “microbead,” etc., (optionally but not necessarily spherical in shape) having a smallest cross-sectional length (e.g., diameter) of 50 microns or less, preferably 10 microns or less, 3 microns or less, approximately 1 micron or less, approximately 0.5 microns or less, e.g., approximately 0.1, 0.2, 0.3, or 0.4 microns, or smaller (e.g., under 1 nanometer, about 1-10 nanometer, about 10-100 nanometers, or about 100-500 nanometers). In an example, the support is at least 0.1 microns. Microparticles or bead supports may be made of a variety of inorganic or organic materials including, but not limited to, glass (e.g., controlled pore glass), silica, zirconia, cross-linked polystyrene, polyacrylate, polymethylmethacrylate, titanium dioxide, latex, polystyrene, etc. Magnetization can facilitate collection and concentration of the microparticle-attached reagents (e.g., polynucleotides or ligases) after amplification, and can also facilitate additional steps (e.g., washes, reagent removal, etc.). In certain embodiments, a population of microparticles having different shapes sizes or colors is used. The microparticles can optionally be encoded, e.g., with quantum dots such that each microparticle or group of microparticles can be individually or uniquely identified.

In some embodiments, a bead support is functionalized for attaching a population of first primers. In some embodiments, a bead is any size that can fit into a reaction chamber. For example, one bead can fit in a reaction chamber. In some embodiments, more than one bead fit in a reaction chamber. In some embodiments, the smallest cross-sectional length of a bead (e.g., diameter) is about 50 microns or less, or about 10 microns or less, or about 3 microns or less, approximately 1 micron or less, approximately 0.5 microns or less, e.g., approximately 0.1, 0.2, 0.3, or 0.4 microns, or smaller (e.g., under 1 nanometer, about 1-10 nanometer, about 10-100 nanometers, or about 100-500 nanometers).

In general, the bead support can be treated to include a biomolecule, including nucleosides, nucleotides, nucleic acids (oligonucleotides and polynucleotides), polypeptides, saccharides, polysaccharides, lipids, or derivatives or analogs thereof. For example, a polymeric particle can bind or attach to a biomolecule. A terminal end or any internal portion of a biomolecule can bind or attach to a polymeric particle. A polymeric particle can bind or attach to a biomolecule using linking chemistries. A linking chemistry includes covalent or non-covalent bonds, including an ionic bond, hydrogen bond, affinity bond, dipole-dipole bond, van der Waals bond, and hydrophobic bond. A linking chemistry includes affinity between binding partners, for example between: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; or an oligonucleotide or polynucleotide and its corresponding complement.

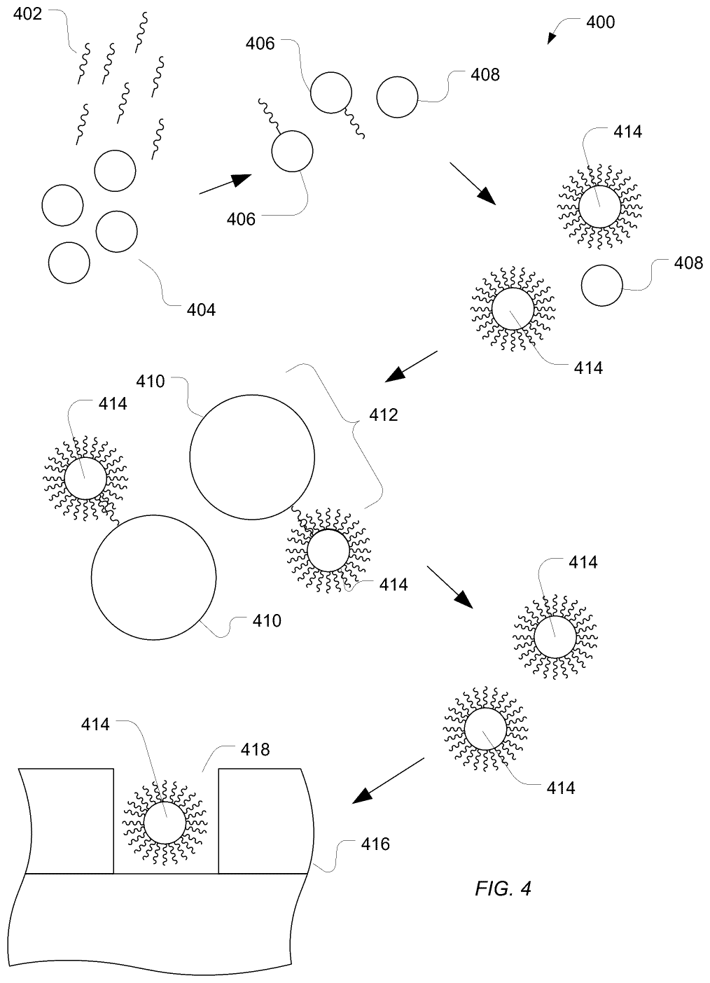

FIG. 4 includes an illustration of an example method for forming nucleic acid supports and loading such supports onto a substrate for sequencing. A plurality of target nucleic acids 402 and supports 404 are provided in solution. Some of the target nucleic acids bind with the supports 406 while other supports 408 remain free of target nucleic acids. Target nucleic acids can be derived from one or more DNA or RNA samples, such as genomic DNA from different subjects. The target nucleic acids from each sample can be combined and undergo the process described below. Alternatively, the target nucleic acids from each sample can be processed separately.

In some embodiments, the template nucleic acid molecules (template polynucleotides or target polynucleotides) can be derived from a sample that can be from a natural or non-natural source. The nucleic acid molecules in the sample can be derived from a living organism or a cell. Any nucleic acid molecule can be used, for example, the sample can include genomic DNA covering a portion of or an entire genome, mRNA, or miRNA from the living organism or cell. In other embodiments, the template nucleic acid molecules can be synthetic or recombinant. In some embodiments, the sample contains nucleic acid molecules having substantially identical sequences or having a mixture of different sequences. Illustrative embodiments are typically performed using nucleic acid molecules that were generated within and by a living cell. Such nucleic acid molecules are typically isolated directly from a natural source, such as a cell or a bodily fluid without any in vitro amplification. Accordingly, the sample nucleic acid molecules are used directly in subsequent steps. In some embodiments, the nucleic acid molecules in the sample can include two or more nucleic acid molecules with different sequences.

The methods can optionally include a target enrichment step before, during, or after the library preparation and before a seeding reaction. Target nucleic acid molecules, including target loci or regions of interest, can be enriched, for example, through multiplex nucleic acid amplification or hybridization. A variety of methods can be used to perform multiplex nucleic acid amplification to generate amplicons, such as multiplex PCR, and can be used in an embodiment. Enrichment by any method can be followed by a universal amplification reaction before the template nucleic acid molecules are added to a seeding reaction mixture. Any of the embodiments of the present teachings can include enriching a plurality of at least 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 400, 500, 600, 700, 800, 900, 1,000, 2,000, 3,000, 4,000, 5,000, 6,000, 7,000, 8,000, 9,000, or 10,000 target nucleic acid molecules, target loci, or regions of interest. In any of the disclosed embodiments, the target loci or regions of interest can be at least 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, 500, 600, 700, 800, 900, or 1,000 nucleotides in length and include a portion of or the entirety of the template nucleic acid molecule. In other embodiments, the target loci or regions of interest can be between about 1 and 10,000 nucleotides in length, for example between about 2 and 5,000 nucleotides, between about 2 and 3,000 nucleotides, or between about 2 and 2,000 nucleotides in length. In any of the embodiments of the present teachings, the multiplex nucleic acid amplification can include generating at least 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 400, 500, 600, 700, 800, 900, 1,000, 2,000, 3,000, 4,000, 5,000, 6,000, 7,000, 8,000, 9,000, or 10,000 copies of each target nucleic acid molecule, target locus, or region of interest.

In some embodiments, after the library preparation and optional enrichment step, the library of template nucleic acid molecules can be templated onto one or more supports. The one or more supports can be templated in two reactions, a seeding reaction to generate seeded solid supports and a templating reaction using the one or more seeded supports to further amplify the attached template nucleic acid molecules. The seeding reaction is typically an amplification reaction and can be performed using a variety of methods. For example, the seeding reaction can be performed in an RPA reaction, a template walking reaction, or a PCR. In an RPA reaction, template nucleic acid molecules are amplified using a recombinase, polymerase, and optionally a recombinase accessory protein in the presence of primers and nucleotides. The recombinase and optionally the recombinase accessory protein can dissociate at least a portion of a double stranded template nucleic acid molecules to allow primers to hybridize that the polymerase can then bind to initiate replication. In some embodiments, the recombinase accessory protein can be a single-stranded binding protein (SSB) that prevents the re-hybridization of dissociated template nucleic acid molecules. Typically, RPA reactions can be performed at isothermal temperatures. In a template walking reaction, template nucleic acid molecules are amplified using a polymerase in the presence of primers and nucleotides in reaction conditions that allow at least a portion of double-stranded template nucleic acid molecules to dissociate such that primers can hybridize, and the polymerase can then bind to initiate replication. In PCR, the double-stranded template nucleic acid molecules are dissociated by thermal cycling. After cooling, primers bind to complementary sequences and can be used for replication by the polymerase. In any of the aspects of the present teachings, the seeding reaction can be performed in a seeding reaction mixture, which is formed with the components necessary for amplification of the template nucleic acid molecules. In any of the disclosed aspects, the seeding reaction mixture can include some or all of the following: a population of template nucleic acid molecules, a polymerase, one or more solid supports with a population of attached first primers, nucleotides, and a cofactor such as a divalent cation. In some embodiments, the seeding reaction mixture can further include a second primer and optionally a diffusion-limiting agent. In some embodiments, the population of template nucleic acid molecules comprise template nucleic acid molecules joined to at least one adaptor sequence which can hybridize to the first or second primers. In some embodiments, the reaction mixture can form an emulsion, as in emulsion RPA or emulsion PCR. In seeding reactions carried out by RPA reactions, the seeding reaction mixture can include a recombinase and optionally a recombinase accessory protein. The various components of the reaction mixture are discussed in further detail herein.

The target nucleic acid secured to the supports 406 are amplified to form multiple copies of the target nucleic acids on the supports 414. Supports 408 remain free of target nucleic acids. Amplification can include a polymerase chain reaction (PCR) amplification, such as emulsion PCR, or isothermal amplification in an emulsion or in bulk solution, such as recombinase-polymerase amplification (RPA).

Capture primers having a capture moiety and complementary to terminal ends of the target nucleic acids secured to the supports can be used to bind the supports 414 to magnetic beads 410 having a moiety that binds with the capture moiety. In an example capture moiety is biotin which binds to streptavidin, for example. As illustrated, the supports 414 including target nucleic acids combined with magnetic beads 410 to form a complex 412. A magnetic field can be used to secure the magnetic beads 410 and the supports 414 including target nucleic acids while the remaining solution is washed away, including supports 408 free of target nucleic acid.

Magnetic beads (e.g., Dynabeads from Dynal, Oslo, Norway) can have a size in a range of 1 micron to 100 microns, such as 2 microns to 100 microns. The magnetic beads can be formed of inorganic or organic materials including, but not limited to, glass (e.g., controlled pore glass), silica, zirconia, cross-linked polystyrene, polystyrene, or a combination thereof.

The supports 414 including target nucleic acids can be separated from the magnetic beads 410 by denaturing or de-hybridizing the capture moiety from the target nucleic acid. For example, a change in ion concentration or an increase in temperature may result in the release of the capture primer from the target nucleic acid. Using a magnetic field, the magnetic beads 410 can be separated from the supports 414 including target nucleic acids.

The supports 414 including the target nucleic acid can be applied to the substrate such as substrate 416. In an example, the substrate includes a set of wells 418 into which the supports 414 are deposited. Sequencing can be performed using the supports including target nucleic acid, for example by sequencing-by-synthesis.

In an example, a sequencing primer can be added to the wells 418 or the bead support 414 can be pre-exposed to the primer prior to placement in the well 418. In particular, the bead support 414 can include bound sequencing primer. The sequencing primer and polynucleotide form a nucleic acid duplex including the polynucleotide (e.g., a template nucleic acid) hybridized to the sequencing primer. The nucleic acid duplex is an at least partially double-stranded polynucleotide. Enzymes and nucleotides can be provided to the well 418 to facilitate detectible reactions, such as nucleotide incorporation.

In a particular embodiment, an enzyme such as a polymerase is present, bound to, or is in close proximity to the particles or beads. In an example, a polymerase is present in solution or in the well to facilitate duplication of the polynucleotide. A variety of nucleic acid polymerase may be used in the methods described herein. In an example embodiment, the polymerase can include an enzyme, fragment, or subunit thereof, which can catalyze duplication of the polynucleotide. In another embodiment, the polymerase can be a naturally occurring polymerase, recombinant polymerase, mutant polymerase, variant polymerase, fusion or otherwise engineered polymerase, chemically modified polymerase, synthetic molecules, or analog, derivative, or fragment thereof. Example enzymes, solutions, compositions, and amplification methods can be found in WO2019/094,524, titled “METHODS AND COMPOSITIONS FOR MANIPULATING NUCLEIC ACIDS”, which is incorporated herein by reference in its entirety.

Sequencing can be performed by detecting nucleotide addition. Nucleotide addition can be detected using methods such as fluorescent emission methods or ion detection methods. For example, a set of fluorescently labeled nucleotides can be provided to the system 416 and can migrate to the well 418. Excitation energy can be also provided to the well 418. When a nucleotide is captured by a polymerase and added to the end of an extending primer, a label of the nucleotide can fluoresce, indicating which type of nucleotide is added.

In an alternative example, solutions including a single type of nucleotide can be fed sequentially. In response to nucleotide addition, the pH within the local environment of the well 418 can change. Such a change in pH can be detected by ion sensitive field effect transistors (ISFET). As such, a change in pH can be used to generate a signal indicating the order of nucleotides complementary to the polynucleotide of the particle 410.

In particular, a sequencing system can include a well, or a plurality of wells, disposed over a sensor pad of an ionic sensor, such as a field effect transistor (FET). In embodiments, a system includes one or more polymeric particles loaded into a well which is disposed over a sensor pad of an ionic sensor (e.g., FET), or one or more polymeric particles loaded into a plurality of wells which are disposed over sensor pads of ionic sensors (e.g., FET). In embodiments, an FET can be a chemFET or an ISFET. A “chemFET” or chemical field-effect transistor, includes a type of field effect transistor that acts as a chemical sensor. The chemFET has the structural analog of a MOSFET transistor, where the charge on the gate electrode is applied by a chemical process. An “ISFET” or ion-sensitive field-effect transistor, can be used for measuring ion concentrations in solution; when the ion concentration (such as H+) changes, the current through the transistor changes accordingly.

In embodiments, the FET may be a FET array. As used herein, an “array” is a planar arrangement of elements such as sensors or wells. The array may be one or two dimensional. A one-dimensional array can be an array having one column (or row) of elements in the first dimension and a plurality of columns (or rows) in the second dimension. The number of columns (or rows) in the first and second dimensions may or may not be the same. The FET or array can comprise 102, 103, 104, 105, 106, 107 or more FETs.

In embodiments, one or more microfluidic structures can be fabricated above the FET sensor array to provide for containment or confinement of a biological or chemical reaction. For example, in one implementation, the microfluidic structure(s) can be configured as one or more wells (or wells, or reaction chambers, or reaction wells, as the terms are used interchangeably herein) disposed above one or more sensors of the array, such that the one or more sensors over which a given well is disposed detect and measure analyte presence, level, or concentration in the given well. In embodiments, there can be a 1:1 correspondence of FET sensors and reaction wells.

Returning to FIG. 4, in another example, a well 418 of the array of wells can be operatively connected to measuring devices. For example, for fluorescent emission methods, a well 418 can be operatively coupled to a light detection device. In the case of ionic detection, the lower surface of the well 418 may be disposed over a sensor pad of an ionic sensor, such as a field effect transistor.

One example system involving sequencing via detection of ionic byproducts of nucleotide incorporation is the Ion Torrent PGM™, Proton™, S5™ or Genexus™ sequencer (Thermo Fisher Scientific), which is an ion-based sequencing system that sequences nucleic acid templates by detecting hydrogen ions produced as a byproduct of nucleotide incorporation. Typically, hydrogen ions are released as byproducts of nucleotide incorporations occurring during template-dependent nucleic acid synthesis by a polymerase. The Ion Torrent PGM™, Proton™, S5™ or Genexus™ sequencer detects the nucleotide incorporations by detecting the hydrogen ion byproducts of the nucleotide incorporations. The Ion Torrent PGM™, Proton™, S5™ or Genexus™ sequencer can include a plurality of template polynucleotides to be sequenced, each template disposed within a respective sequencing reaction well in an array. The wells of the array can each be coupled to at least one ion sensor that can detect the release of H+ ions or changes in solution pH produced as a byproduct of nucleotide incorporation. The ion sensor comprises a field effect transistor (FET) coupled to an ion-sensitive detection layer that can sense the presence of H+ ions or changes in solution pH. The ion sensor can provide output signals indicative of nucleotide incorporation which can be represented as voltage changes whose magnitude correlates with the H+ ion concentration in a respective well or reaction chamber. Different nucleotide types can be flowed serially into the reaction chamber and can be incorporated by the polymerase into an extending primer (or polymerization site) in an order determined by the sequence of the template. Each nucleotide incorporation can be accompanied by the release of H+ ions in the reaction well, along with a concomitant change in the localized pH. The release of H+ ions can be registered by the FET of the sensor, which produces signals indicating the occurrence of the nucleotide incorporation. Nucleotides that are not incorporated during a particular nucleotide flow may not produce signals. The amplitude of the signals from the FET can also be correlated with the number of nucleotides of a particular type incorporated into the extending nucleic acid molecule thereby permitting homopolymer regions to be resolved. Thus, during a run of the sequencer multiple nucleotide flows into the reaction chamber along with incorporation monitoring across a multiplicity of wells or reaction chambers can permit the instrument to resolve the sequence of many nucleic acid templates simultaneously.

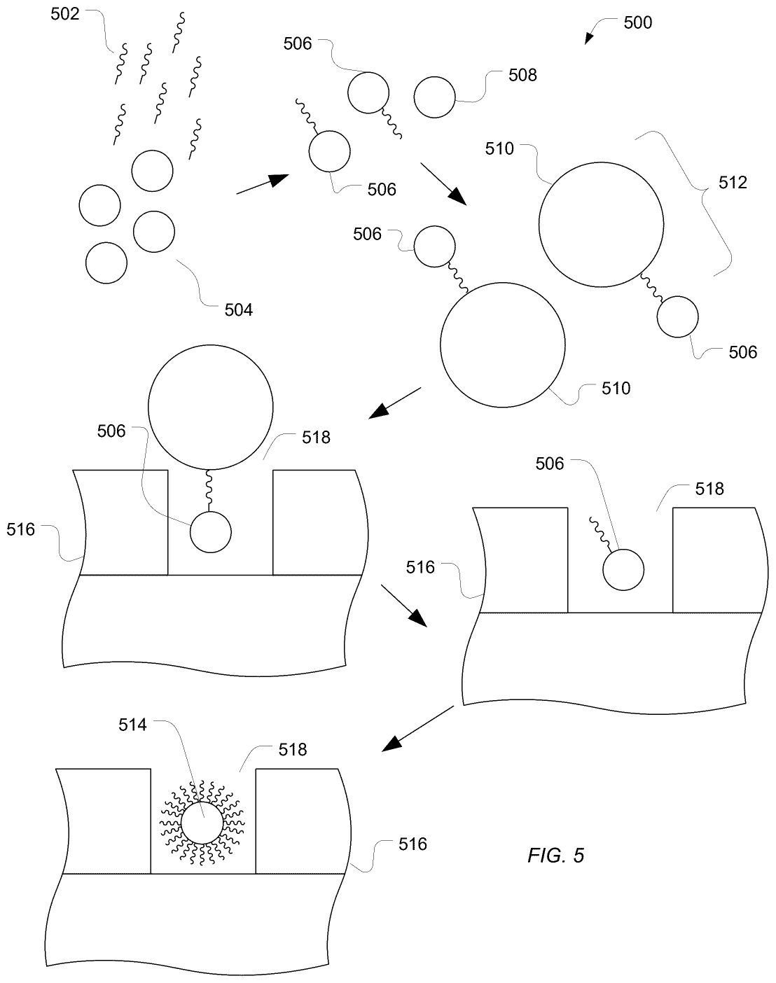

In another example illustrated in FIG. 5, polymeric particles can be used as a support for polynucleotides during sequencing techniques. For example, such hydrophilic particles can immobilize a polynucleotide for sequencing using fluorescent sequencing techniques. In another example, the hydrophilic particles can immobilize a plurality of copies of a polynucleotide for sequencing using ion-sensing techniques. Alternatively, the above described treatments can improve polymer matrix bonding to a surface of a sensor array. The polymer matrices can capture analytes, such as polynucleotides for sequencing.

A bead support may be composed of organic polymers such as polystyrene, polyethylene, polypropylene, polyfluoroethylene, polyethyleneoxy, and polyacrylamide, as well as co-polymers and grafts thereof. A support may also be inorganic, such as glass, silica, controlled-pore-glass (CPG), or reverse-phase silica. The configuration of a support may be in the form of beads, spheres, particles, granules, a gel, or a surface. Supports may be porous or non-porous and may have swelling or non-swelling characteristics. In some embodiments, a support is an Ion Sphere Particle. Example bead supports are disclosed in U.S. Pat. No. 9,243,085, titled “Hydrophilic Polymeric Particles and Methods for Making and Using Same,” and in U.S. Pat. No. 9,868,826, titled “Polymer Substrates Formed from Carboxy Functional Acrylamide,” each of which is incorporated herein by reference.

In some embodiments, the solid support is a “microparticle,” “bead,” “microbead,” etc., (optionally but not necessarily spherical in shape) having a smallest cross-sectional length (e.g., diameter) of 50 microns or less, preferably 10 microns or less, 3 microns or less, approximately 1 micron or less, approximately 0.5 microns or less, e.g., approximately 0.1, 0.2, 0.3, or 0.4 microns, or smaller (e.g., under 1 nanometer, about 1-10 nanometer, about 10-100 nanometers, or about 100-500 nanometers). In an example, the support is at least 0.1 microns. Microparticles or bead supports may be made of a variety of inorganic or organic materials including, but not limited to, glass (e.g., controlled pore glass), silica, zirconia, cross-linked polystyrene, polyacrylate, polymethylmethacrylate, titanium dioxide, latex, polystyrene, etc. Magnetization can facilitate collection and concentration of the microparticle-attached reagents (e.g., polynucleotides or ligases) after amplification, and can also facilitate additional steps (e.g., washes, reagent removal, etc.). In certain embodiments, a population of microparticles having different shapes sizes or colors is used. The microparticles can optionally be encoded, e.g., with quantum dots such that each microparticle or group of microparticles can be individually or uniquely identified.

Magnetic beads (e.g., Dynabeads from Dynal, Oslo, Norway) can have a size in a range of 1 micron to 100 microns, such as 2 microns to 100 microns. The magnetic beads can be formed of inorganic or organic materials including, but not limited to, glass (e.g., controlled pore glass), silica, zirconia, cross-linked polystyrene, polystyrene, or a combination thereof.

In some embodiments, a bead support is functionalized for attaching a population of first primers. In some embodiments, a bead is any size that can fit into a reaction chamber. For example, one bead can fit in a reaction chamber. In some embodiments, more than one bead fit in a reaction chamber. In some embodiments, the smallest cross-sectional length of a bead (e.g., diameter) is about 50 microns or less, or about 10 microns or less, or about 3 microns or less, approximately 1 micron or less, approximately 0.5 microns or less, e.g., approximately 0.1, 0.2, 0.3, or 0.4 microns, or smaller (e.g., under 1 nanometer, about 1-10 nanometer, about 10-100 nanometers, or about 100-500 nanometers).

In general, the bead support can be treated to include a biomolecule, including nucleosides, nucleotides, nucleic acids (oligonucleotides and polynucleotides), polypeptides, saccharides, polysaccharides, lipids, or derivatives or analogs thereof. For example, a polymeric particle can bind or attach to a biomolecule. A terminal end or any internal portion of a biomolecule can bind or attach to a polymeric particle. A polymeric particle can bind or attach to a biomolecule using linking chemistries. A linking chemistry includes covalent or non-covalent bonds, including an ionic bond, hydrogen bond, affinity bond, dipole-dipole bond, van der Waals bond, and hydrophobic bond. A linking chemistry includes affinity between binding partners, for example between: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; or an oligonucleotide or polynucleotide and its corresponding complement.

As illustrated in FIG. 5, a plurality of bead supports 504 can be placed in a solution along with a plurality of polynucleotides 502 (target or template polynucleotides). Target or template polynucleotides can be derived from one or more DNA or RNA samples, such as genomic DNA from different subjects. The target or template polynucleotides from each sample can be combined and undergo the process described below. Alternatively, the target or template polynucleotides from each sample can be processed separately.

The plurality of bead supports 504 can be activated or otherwise prepared to bind with the polynucleotides 502. For example, the bead supports 504 can include an oligonucleotide (capture primer) complementary to a portion of a polynucleotide of the plurality of polynucleotides 502. In another example, the bead supports 504 can be modified with target polynucleotides 502 using techniques such as biotin-streptavidin binding.

In some embodiments, the template nucleic acid molecules (template polynucleotides or target polynucleotides) can be derived from a sample that can be from a natural or non-natural source. The nucleic acid molecules in the sample can be derived from a living organism or a cell. Any nucleic acid molecule can be used, for example, the sample can include genomic DNA covering a portion of or an entire genome, mRNA, or miRNA from the living organism or cell. In other embodiments, the template nucleic acid molecules can be synthetic or recombinant. In some embodiments, the sample contains nucleic acid molecules having substantially identical sequences or having a mixture of different sequences. Illustrative embodiments are typically performed using nucleic acid molecules that were generated within and by a living cell. Such nucleic acid molecules are typically isolated directly from a natural source such as a cell or a bodily fluid without any in vitro amplification. Accordingly, the sample nucleic acid molecules are used directly in subsequent steps. In some embodiments, the nucleic acid molecules in the sample can include two or more nucleic acid molecules with different sequences.

The methods can optionally include a target enrichment step before, during, or after the library preparation and before a seeding reaction. Target nucleic acid molecules, including target loci or regions of interest, can be enriched, for example, through multiplex nucleic acid amplification or hybridization. A variety of methods can be used to perform multiplex nucleic acid amplification to generate amplicons, such as multiplex PCR, and can be used in an embodiment. Enrichment by any method can be followed by a universal amplification reaction before the template nucleic acid molecules are added to a seeding reaction mixture. Any of the embodiments of the present teachings can include enriching a plurality of at least 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 400, 500, 600, 700, 800, 900, 1,000, 2,000, 3,000, 4,000, 5,000, 6,000, 7,000, 8,000, 9,000, or 10,000 target nucleic acid molecules, target loci, or regions of interest. In any of the disclosed embodiments, the target loci or regions of interest can be at least 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, 500, 600, 700, 800, 900, or 1,000 nucleotides in length and include a portion of or the entirety of the template nucleic acid molecule. In other embodiments, the target loci or regions of interest can be between about 1 and 10,000 nucleotides in length, for example between about 2 and 5,000 nucleotides, between about 2 and 3,000 nucleotides, or between about 2 and 2,000 nucleotides in length. In any of the embodiments of the present teachings, the multiplex nucleic acid amplification can include generating at least 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 400, 500, 600, 700, 800, 900, 1,000, 2,000, 3,000, 4,000, 5,000, 6,000, 7,000, 8,000, 9,000, or 10,000 copies of each target nucleic acid molecule, target locus, or region of interest.

In some embodiments, after the library preparation and optional enrichment step, the library of template nucleic acid molecules can be templated onto one or more supports. The one or more supports can be templated in two reactions, a seeding reaction to generate seeded solid supports and a templating reaction using the one or more seeded supports to further amplify the attached template nucleic acid molecules. The seeding reaction is typically an amplification reaction and can be performed using a variety of methods. For example, the seeding reaction can be performed in an RPA reaction, a template walking reaction, or a PCR. In an RPA reaction, template nucleic acid molecules are amplified using a recombinase, polymerase, and optionally a recombinase accessory protein in the presence of primers and nucleotides. The recombinase and optionally the recombinase accessory protein can dissociate at least a portion of a double stranded template nucleic acid molecules to allow primers to hybridize that the polymerase can then bind to initiate replication. In some embodiments, the recombinase accessory protein can be a single-stranded binding protein (SSB) that prevents the re-hybridization of dissociated template nucleic acid molecules. Typically, RPA reactions can be performed at isothermal temperatures. In a template walking reaction, template nucleic acid molecules are amplified using a polymerase in the presence of primers and nucleotides in reaction conditions that allow at least a portion of double-stranded template nucleic acid molecules to dissociate such that primers can hybridize, and the polymerase can then bind to initiate replication. In PCR, the double-stranded template nucleic acid molecules are dissociated by thermal cycling. After cooling, primers bind to complementary sequences and can be used for replication by the polymerase. In any of the aspects of the present teachings, the seeding reaction can be performed in a seeding reaction mixture, which is formed with the components necessary for amplification of the template nucleic acid molecules. In any of the disclosed aspects, the seeding reaction mixture can include some or all of the following: a population of template nucleic acid molecules, a polymerase, one or more solid supports with a population of attached first primers, nucleotides, and a cofactor such as a divalent cation. In some embodiments, the seeding reaction mixture can further include a second primer and optionally a diffusion-limiting agent. In some embodiments, the population of template nucleic acid molecules comprise template nucleic acid molecules joined to at least one adaptor sequence which can hybridize to the first or second primers. In some embodiments, the reaction mixture can form an emulsion, as in emulsion RPA or emulsion PCR. In seeding reactions carried out by RPA reactions, the seeding reaction mixture can include a recombinase and optionally a recombinase accessory protein. The various components of the reaction mixture are discussed in further detail herein.

In a particular embodiment of seeding, the hydrophilic particles and polynucleotides are subjected to polymerase chain reaction (PCR) amplification or recombinase polymerase amplification (RPA). In an example, the particles 504 include a capture primer complementary to a portion of the template polynucleotide 502. The template polynucleotide can hybridize to the capture primer. The capture primer can be extended to form beads 506 that include a target polynucleotide attached thereto. Other beads may remain unattached to a target nucleic acid and other template polynucleotide can be free floating in solution.

In an example, the bead support 506 including a target polynucleotide can be attached to a magnetic bead 510 to form a bead assembly 512. In particular, the magnetic bead 510 is attached to the bead support 506 by a double stranded polynucleotide linkage. In an example, a further probe including a linker moiety can hybridize to a portion of the target polynucleotide on the bead support 506. The linker moiety can be attached to a complementary linker moiety on the magnetic bead 510. In another example, the template polynucleotide used to form the target nucleic acid attached to beads 506 can include a linker moiety that attaches to the magnetic bead 510. In another example, the template polynucleotide complementary to target polynucleotide attached to the bead support 506 can be generated from a primer that is modified with a linker that attaches to the magnetic bead 510.

The linker moiety attached to the polynucleotide and the linker moiety attached to the magnetic bead can be complementary to and attach to each other. In an example, the linker moieties have affinity and can include: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; or an oligonucleotide or polynucleotide and its corresponding complement. In a particular example, the linker moiety attached to the polynucleotide includes biotin and the linker moiety attached to the magnetic bead includes streptavidin.

The bead assemblies 512 can be applied over a substrate 516 of a sequencing device that includes wells 518. In an example, a magnetic field can be applied to the substrate 516 to draw the magnetic beads 510 of the bead assembly 512 towards the wells 518. The bead support 506 enters the well 518. For example, a magnet can be moved in parallel to a surface of the substrate 516 resulting in the deposition of the bead support 406 in the wells 518.

The bead assembly 512 can be denatured to remove the magnetic bead 510 leaving the bead support 506 in the well 518. For example, hybridized double-stranded DNA of the bead assembly 512 can be denatured using thermal cycling or ionic solutions to release the magnetic bead 510 and template polynucleotides having a linker moiety attached to the magnetic bead 510. For example, the double-stranded DNA can be treated with low ion-content aqueous solutions, such as deionized water, to denature and separate the strands. In an example, a foam wash can be used to remove the magnetic beads.

Optionally, the target polynucleotides 506 can be amplified, referred to herein as templating, while in the well 518, to provide a bead support 514 with multiple copies of the target polynucleotides. In particular, the bead 514 has a monoclonal population of target polynucleotides. Such an amplification reactions can be performed using polymerase chain reaction (PCR) amplification, recombination polymerase amplification (RPA) or a combination thereof. Alternatively, amplification can be performed prior to depositing the bead support 514 in the well.

In a particular embodiment, an enzyme such as a polymerase is present, bound to, or is in close proximity to the particles or beads. In an example, a polymerase is present in solution or in the well to facilitate duplication of the polynucleotide. A variety of nucleic acid polymerase may be used in the methods described herein. In an example embodiment, the polymerase can include an enzyme, fragment, or subunit thereof, which can catalyze duplication of the polynucleotide. In another embodiment, the polymerase can be a naturally occurring polymerase, recombinant polymerase, mutant polymerase, variant polymerase, fusion or otherwise engineered polymerase, chemically modified polymerase, synthetic molecules, or analog, derivative, or fragment thereof. Example enzymes, solutions, compositions, and amplification methods can be found in WO2019/094,524, titled “METHODS AND COMPOSITIONS FOR MANIPULATING NUCLEIC ACIDS”, which is incorporated herein by reference in its entirety.

While the polynucleotides of bead support 514 are illustrated as being on a surface, the polynucleotides can extend within the bead support 514. Hydrogel and hydrophilic particles having a low concentration of polymer relative to water can include polynucleotide segments on the interior of and throughout the bead support 514 or polynucleotides can reside in pores and other openings. In particular, the bead support 514 can permit diffusion of enzymes, nucleotides, primers, and reaction products used to monitor the reaction. A high number of polynucleotides per particle produces a better signal.

In an example embodiment, the bead support 514 can be utilized in a sequencing device. For example, a sequencing device 516 can include an array of wells 518.

In an example, a sequencing primer can be added to the wells 518 or the bead support 514 can be pre-exposed to the primer prior to placement in the well 518. In particular, the bead support 514 can include bound sequencing primer. The sequencing primer and polynucleotide form a nucleic acid duplex including the polynucleotide (e.g., a template nucleic acid) hybridized to the sequencing primer. The nucleic acid duplex is an at least partially double-stranded polynucleotide. Enzymes and nucleotides can be provided to the well 518 to facilitate detectible reactions, such as nucleotide incorporation.

Sequencing can be performed by detecting nucleotide addition. Nucleotide addition can be detected using methods such as fluorescent emission methods or ion detection methods. For example, a set of fluorescently labeled nucleotides can be provided to the system 516 and can migrate to the well 518. Excitation energy can be also provided to the well 518. When a nucleotide is captured by a polymerase and added to the end of an extending primer, a label of the nucleotide can fluoresce, indicating which type of nucleotide is added.

In an alternative example, solutions including a single type of nucleotide can be fed sequentially. In response to nucleotide addition, the pH within the local environment of the well 518 can change. Such a change in pH can be detected by ion sensitive field effect transistors (ISFET). As such, a change in pH can be used to generate a signal indicating the order of nucleotides complementary to the polynucleotide of the particle 510.

In particular, a sequencing system can include a well, or a plurality of wells, disposed over a sensor pad of an ionic sensor, such as a field effect transistor (FET). In embodiments, a system includes one or more polymeric particles loaded into a well which is disposed over a sensor pad of an ionic sensor (e.g., FET), or one or more polymeric particles loaded into a plurality of wells which are disposed over sensor pads of ionic sensors (e.g., FET). In embodiments, an FET can be a chemFET or an ISFET. A “chemFET” or chemical field-effect transistor, includes a type of field effect transistor that acts as a chemical sensor. The chemFET has the structural analog of a MOSFET transistor, where the charge on the gate electrode is applied by a chemical process. An “ISFET” or ion-sensitive field-effect transistor, can be used for measuring ion concentrations in solution; when the ion concentration (such as H+) changes, the current through the transistor changes accordingly.

In embodiments, the FET may be a FET array. As used herein, an “array” is a planar arrangement of elements such as sensors or wells. The array may be one or two dimensional. A one-dimensional array can be an array having one column (or row) of elements in the first dimension and a plurality of columns (or rows) in the second dimension. The number of columns (or rows) in the first and second dimensions may or may not be the same. The FET or array can comprise 102, 103, 104, 105, 106, 107 or more FETs.

In embodiments, one or more microfluidic structures can be fabricated above the FET sensor array to provide for containment or confinement of a biological or chemical reaction. For example, in one implementation, the microfluidic structure(s) can be configured as one or more wells (or wells, or reaction chambers, or reaction wells, as the terms are used interchangeably herein) disposed above one or more sensors of the array, such that the one or more sensors over which a given well is disposed detect and measure analyte presence, level, or concentration in the given well. In embodiments, there can be a 1:1 correspondence of FET sensors and reaction wells.

Returning to FIG. 5, in another example, a well 518 of the array of wells can be operatively connected to measuring devices. For example, for fluorescent emission methods, a well 518 can be operatively coupled to a light detection device. In the case of ionic detection, the lower surface of the well 418 may be disposed over a sensor pad of an ionic sensor, such as a field effect transistor.

One example system involving sequencing via detection of ionic byproducts of nucleotide incorporation is the Ion Torrent PGM™, Proton™, S5™ or Genexus™ sequencer (Thermo Fisher Scientific), which is an ion-based sequencing system that sequences nucleic acid templates by detecting hydrogen ions produced as a byproduct of nucleotide incorporation. Typically, hydrogen ions are released as byproducts of nucleotide incorporations occurring during template-dependent nucleic acid synthesis by a polymerase. The Ion Torrent PGM™, Proton™, S5™ or Genexus™ sequencer detects the nucleotide incorporations by detecting the hydrogen ion byproducts of the nucleotide incorporations. The Ion Torrent PGM™, Proton™, S5™ or Genexus™ sequencer can include a plurality of template polynucleotides to be sequenced, each template disposed within a respective sequencing reaction well in an array. The wells of the array can each be coupled to at least one ion sensor that can detect the release of H+ ions or changes in solution pH produced as a byproduct of nucleotide incorporation. The ion sensor comprises a field effect transistor (FET) coupled to an ion-sensitive detection layer that can sense the presence of H+ ions or changes in solution pH. The ion sensor can provide output signals indicative of nucleotide incorporation which can be represented as voltage changes whose magnitude correlates with the H+ ion concentration in a respective well or reaction chamber. Different nucleotide types can be flowed serially into the reaction chamber and can be incorporated by the polymerase into an extending primer (or polymerization site) in an order determined by the sequence of the template. Each nucleotide incorporation can be accompanied by the release of H+ ions in the reaction well, along with a concomitant change in the localized pH. The release of H+ ions can be registered by the FET of the sensor, which produces signals indicating the occurrence of the nucleotide incorporation. Nucleotides that are not incorporated during a particular nucleotide flow may not produce signals. The amplitude of the signals from the FET can also be correlated with the number of nucleotides of a particular type incorporated into the extending nucleic acid molecule thereby permitting homopolymer regions to be resolved. Thus, during a run of the sequencer multiple nucleotide flows into the reaction chamber along with incorporation monitoring across a multiplicity of wells or reaction chambers can permit the instrument to resolve the sequence of many nucleic acid templates simultaneously.

Following amplification of the target nucleic acids on supports, many of the supports can be monoclonal. But some of the support may not have amplified target nucleic acids and some may be polyclonal. The monoclonal supports can be separated from the polyclonal supports utilizing an enrichment barcode region formed on at least some of the target nucleic acids as follows.

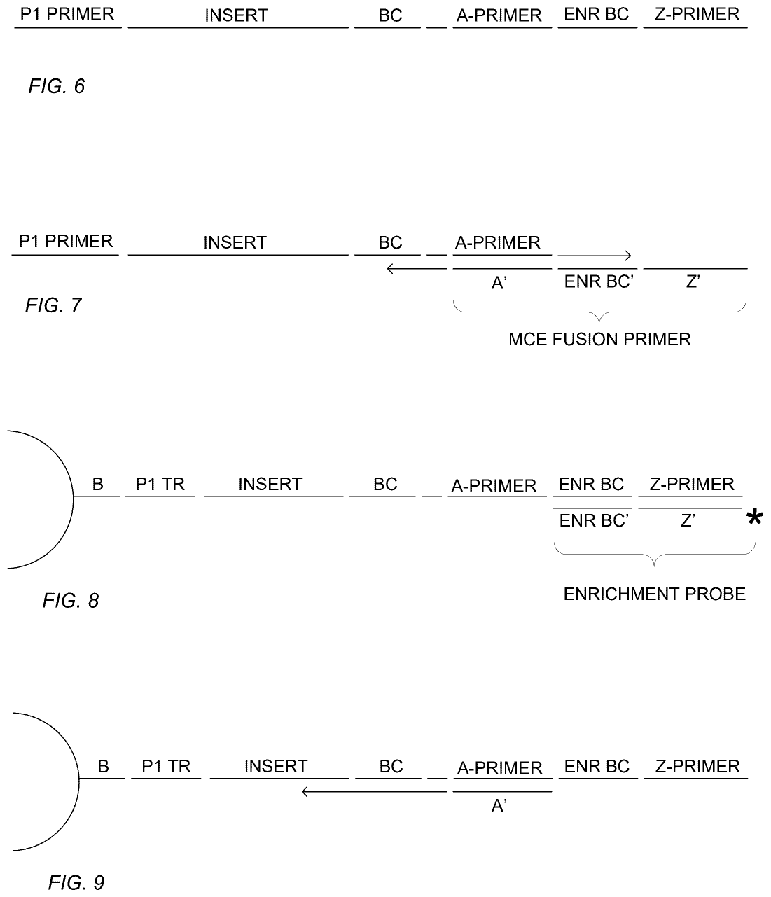

As illustrated in FIG. 6, a target nucleic acid can include an insert region associated with the target sequence. A P1 primer region is formed on 5′ end of the insert region. At 3′ end of the insert region, one or more sequencing barcode regions or key regions can be included. A sequencing primer region (A-Primer) can be included at 3′ end of the barcode regions or key regions. One or more additional regions can be included between the insert region and barcode region or the barcode region and sequencing primer region. At 3′ end of the sequencing primer region, an enrichment section is formed. For example, an enrichment barcode region is formed on 3′ end of the sequencing primer region and on 3′ end of the enrichment barcode region, an enrichment primer region (Z-Primer) is formed. Together, the enrichment barcode region and the enrichment primer region (Z-Primer) are referred to herein as the enrichment section.

In a plurality of target nucleic acids to be amplified on supports, the A-Primer regions and Z-Primer regions can have the same sequence as A-Primer and Z-Primer regions of other target nucleic acids of the plurality, whereas the enrichment barcode regions can be different. For example, the enrichment barcode regions can be different for target nucleic acids derived from different samples or can be different within populations of target nucleic acids derived from the same sample. In an example, samples are derived from different subjects and can be analyzed together. For example, samples can be genomic DNA derived from different test subjects. The enrichment barcode region on a select target nucleic acid can be one sequence of two or more sequences used in forming the plurality of target nucleic acids.

The sequence of the Z-Primer region can include between 5 and 50 nucleotides. For example, the sequence of the Z-Primer region can include between 10 and 30 nucleotides or between 15 and 25 nucleotides. The sequence of the A-Primer region can include between 5 and 50 nucleotides. For example, the sequence of the A-Primer region can include between 10 and 30 nucleotides or between 15 and 25 nucleotides. In an additional example, the sequence of the enrichment barcode region can include between 5 and 50 nucleotides. For example, the sequence of the enrichment barcode region can include between 10 and 30 nucleotides or between 15 and 25 nucleotides.

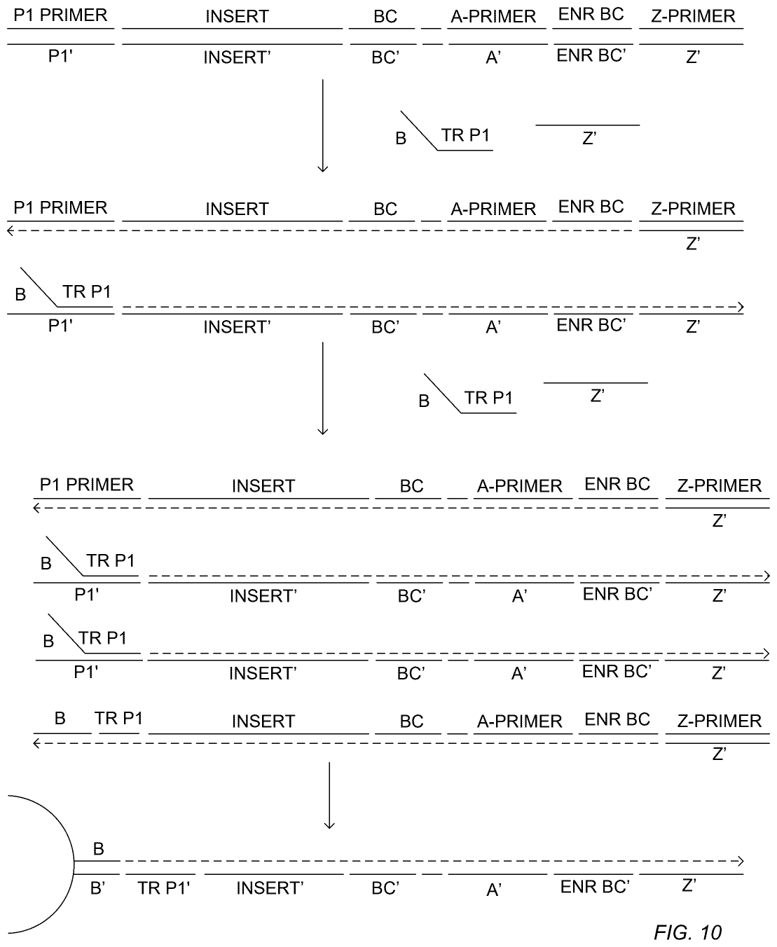

As illustrated in FIG. 7, a target nucleic acid including the target sequence insert can be extended to include an enrichment barcode region and Z-Primer region utilizing a fusion primer (e.g., an MCE fusion primer). The MCE fusion primer includes a region complementary to the A-Primer region, a region complementary to an enrichment barcode region, and a region complementary to the Z-Primer region. In an example, the target nucleic acid sequence can be extended in a single PCR cycle. Alternatively, the target nucleic acid can be extended using an isothermal amplification. The resulting double-stranded nucleic acid can be used in a process to generate a nucleic acid strand that binds to a support. An example process is described in relation to FIG. 10 below.

Target nucleic acids can be derived from different samples, such as genomic DNA of different subjects. The enrichment barcode region and Z-Primer region can be added to target nucleic acids of each sample separately. Alternatively, the enrichment barcode and Z-primer regions can be added to the target nucleic acids of combined samples after the target nucleic acids of the samples are mixed.

Nucleic acid supports can be formed to include the target nucleic acid and can be separated using an enrichment probe or capture probe that is complementary to the enrichment section (e.g., the enrichment barcode and the Z-Primer regions). As illustrated in FIG. 8, the enrichment probe can include a capture moiety. A magnetic bead that includes bound moieties that bind with the capture moiety can be mixed with the nucleic acid supports resulting in nucleic acid supports that include the specific enrichment barcode and Z-Primer region attaching to the magnetic bead. Other nucleic acid supports that include a different enrichment barcode region sequence do not bind with the magnetic bead.

The capture moiety can be one of a pair of binding partners of a linking chemistry. A linking chemistry includes affinity between binding partners, for example between: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; or an oligonucleotide or polynucleotide and its corresponding complement. In an example capture moiety and bound moiety pairs include biotin and streptavidin.

Amplified nucleic acid supports can be used in sequencing applications. For example, the isolated nucleic acid supports can be used for sequencing-by-synthesis methods. As illustrated in the example of FIG. 9, a sequencing primer (A′) complementary to the A-Primer region can be used to synthesize a strand complementary to the target nucleic acid, ignoring the enrichment section (i.e., the enrichment barcode and Z-Primer regions). Accordingly, the enrichment barcode and Z-Primer regions are not sequenced, reducing the amount of reagent used during the sequencing process and reducing the amount of time required to generate sequence data or perform base calling.

Once the target nucleic acid including the enrichment barcode and Z-Primer regions is formed, the target nucleic acid can be amplified on a support to form a population of the target nucleic acids on the support. For example, of the support may include a bound oligonucleotide complementary to the or the same as a portion of the P1 primer region of the target nucleic acid. In another example, the support includes oligonucleotides of another sequence, complements of which can be added to the target nucleic acid to permit binding with the support.

In an example illustrated in FIG. 10, the target nucleic acid including the enrichment barcode region and the Z-Primer regions undergo three polymerize chain reaction (PCR) cycles in the presence of a primer complementary to the Z-Primer region and a primer having a first region the same as a portion (truncated P1 or TR P1) of the P1 primer region and an additional region having a sequence the same as that of the oligonucleotide bound to the support.

After the second PCR cycle, for each target nucleic acid in a set of target nucleic acids, the resulting nucleic acids include a single species that is complementary to the target nucleic acid and has a 3′ region that is complementary to the oligonucleotides secured to the support.

During the third PCR cycle, the oligonucleotide secured to the bead support is extended to form a nucleic acid identical to the target nucleic acid. The supports can be separated from the solution such as through centrifugation or filtration, and the other nucleic acids and primers washed away from the supports. Alternatively, a magnetic separation process can be used. The enrichment probe can include regions complementary to the Z-Primer region and optionally complementary the enrichment barcode region or A-Primer regions. For example, the enrichment probe can have the same sequence as the MCE fusion primer with the addition of a capture moiety.

Supports including target nucleic acids can be amplified to provide multiple copies of the target nucleic acids secured to the support. Amplification can include amplification through polymerase chain reaction (PCR) such as emulsion PCR or can include isothermal amplification using methods such as RPA and bulk solutions. In either case, there exists a possibility that two different target nucleic acids are amplified on the same support, resulting in a portion of the supports being polyclonal. Such polyclonal supports can result in erroneous sequencing data or reduce the amount of useful data acquired during sequencing. Example amplification or templating methods can be found in WO 2020/227031 titled “METHODS AND COMPOSITIONS FOR MANIPULATING NUCLEIC ACIDS.”

Following amplification, monoclonal supports include target nucleic acids having an enrichment barcode region of the same sequence. Polyclonal supports can have target nucleic acids that have different enrichment barcode regions of different sequences.

To separate polyclonal supports from the monoclonal supports, enrichment probes can be used. The enrichment probes have regions complementary to the enrichment section (e.g., the enrichment barcode region or the Z-Primer region) and have a capture moiety, for example, as illustrated in FIG. 8. The enrichment probes include the region complementary to one enrichment barcode region of a set of enrichment barcode sequences. As such, the enrichment probe binds to some of the target nucleic acids having a complementary enrichment barcode region sequence, while not binding to other target nucleotides having a different enrichment barcode region sequence. Magnetic beads having a moiety that binds with the capture moiety can be used to secure supports having target nucleic acids that include the enrichment barcode region associated with the enrichment probe.

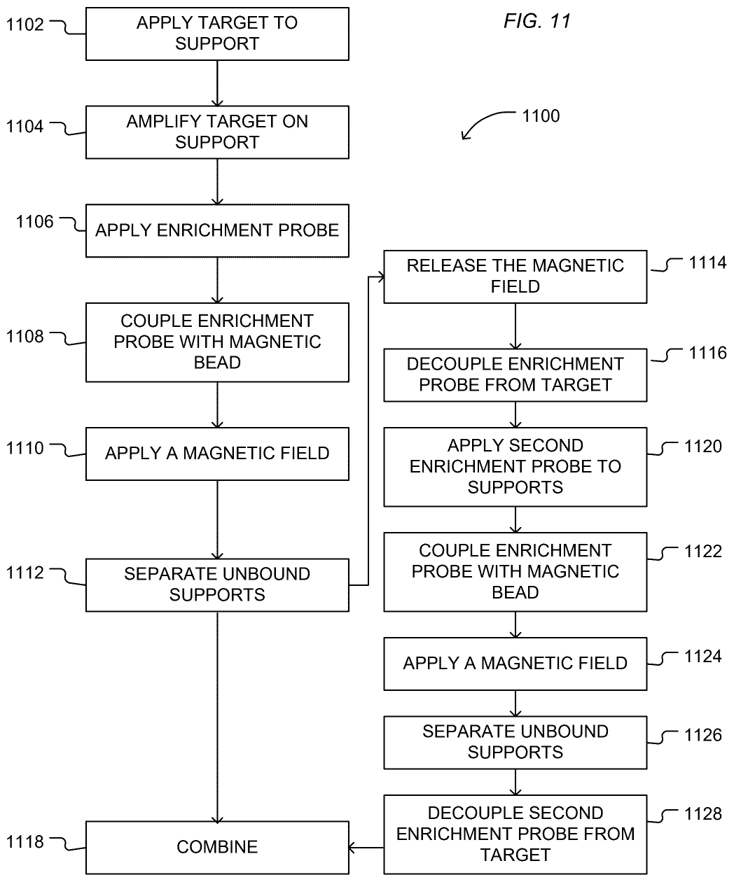

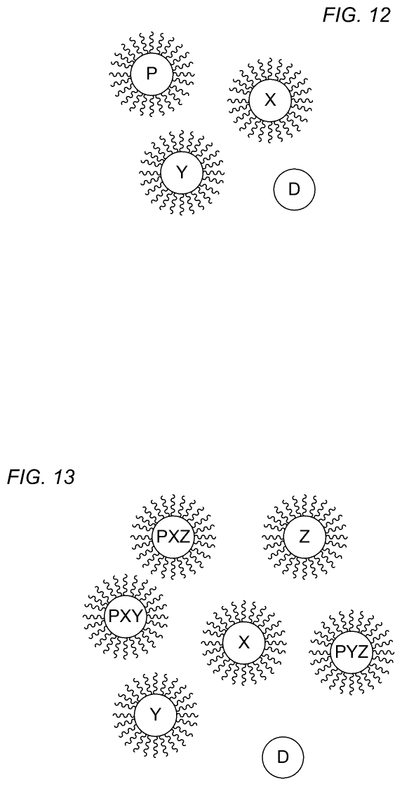

For example, a method illustrated in FIG. 11 utilizes magnetic beads isolate monoclonal supports from polyclonal supports. As illustrated at 1102, target polynucleotides can be bound to supports.

The target polynucleotides can be amplified or templated on the supports, as illustrated at 1104. Amplification or templating can include polymerase chain reaction or isothermal amplification, such as RPA. The amplification can be performed in an emulsion or can be performed in a bulk solution. Alternatively, the following separation can be performed prior to amplification. For example, the separation of polyclonal bead can be performed using the complexes 512, as illustrated in the method of FIG. 5.

As illustrated at 1106, an enrichment probe can be applied in solution with the amplified supports. The population of target polynucleotides can include polynucleotides that have an enrichment barcode region having a sequence of one of two or more types of enrichment barcode region sequences. For example, the population of target polynucleotides can include polynucleotides that have an enrichment barcode first enrichment barcode region and polynucleotides that have an enrichment barcode region of a second sequence different from the first sequence.

The enrichment probe has a region that is complementary to the Z-Primer region and a region that is complementary to one of the enrichment barcode regions of the two or more types of enrichment barcode sequences. The enrichment probe includes a capture moiety that binds to a moiety on the magnetic beads, as illustrated at 1108.

A magnetic field can be applied to the solution, as illustrated at 1110, securing magnetic beads and any supports bound thereto. Any unbounded beads can be separated from the supports secured by the magnetic field. The unbound supports include monoclonal supports (first set of monoclonal supports) that have target nucleic acids with enrichment barcode regions that are not complementary to the enrichment probe.

For the nucleic acid supports secured to the magnetic beads, the magnetic field can be released, as illustrated at 1114. The enrichment probes can then be decoupled from the target nucleic acids of the nucleic acid supports, as illustrated at 1116. Decoupling can result from denaturing using a change in ionic concentration or melt off by raising the temperature.

As illustrated at 1120, a second enrichment probe can be applied to the solution including the second set of monoclonal supports and the polyclonal supports. The second enrichment probe can include a region complementary to the other enrichment barcode sequence of the two or more types of enrichment barcode sequences. Such a second enrichment probe binds to polyclonal supports having target nucleic acids including an enrichment region of the other enrichment barcode sequence and do not bind to monoclonal supports (second set of monoclonal supports) having target nucleic acids with an enrichment barcode region of the one enrichment barcode region sequence.

Magnetic beads having a moiety that binds to the enrichment probe are added, as illustrated at 1122. The magnetic beads form a complex with supports bound to the enrichment probe. As illustrated at 1124, a magnetic field can be applied, and unbound beads can be separated from the nucleic acid supports secured by the magnetic field. In an example, the unbound nucleic acid supports include monoclonal supports (second set of monoclonal supports) having target nucleic acids with a barcode region of the one enrichment barcode region sequence.

The first set of monoclonal supports can be combined with the second set of monoclonal supports, as illustrated at 1118, resulting in a population of nucleic acid supports with an increased concentration of monoclonal supports and a decreased concentration of polyclonal supports.

Following enrichment of the monoclonal supports, the nucleic acid supports can be utilized in sequencing. For example, the isolated nucleic acid supports can be used for sequencing-by-synthesis methods. As illustrated in FIG. 9, a sequencing primer (A′) complementary to the A-Primer region can be used to synthesize a strand complementary to the target nucleic acid, ignoring the enrichment barcode and Z-Primer regions. Accordingly, the enrichment barcode and Z-Primer regions are not sequenced, reducing the amount of reagent used during the sequencing process and reducing the amount of time required to generate sequence data or perform base calling.

When enrichment fusion primers are used that have two different or barcode regions of two different enrichment barcode region sequences, the resulting population of amplified supports nucleic acid supports can include a set of monoclonal supports with target nucleic acids or target polynucleotides having an enrichment barcode region of the first sequence, can include a set of monoclonal supports with target nucleic acids or target polynucleotides having an enrichment barcode region of the second sequence, and can include a set of polyclonal supports having target nucleic acids having both target nucleic acids with enrichment regions of the first sequence and target nucleic acids with enrichment regions of the second sequence. For example, as illustrated in FIG. 12, the population of nucleic acid supports can include a set of monoclonal supports having a target nucleic acid with an enrichment region of a first sequence (X), a second set of monoclonal supports having a target nucleic acid with an enrichment region of the second sequence (Y), and a set of polyclonal supports (P) having both target nucleic acids with enrichment regions the first sequence and target nucleic acids with enrichment regions of the second sequence. The population may also include dud beads that do not include or have target nucleic acids (D).

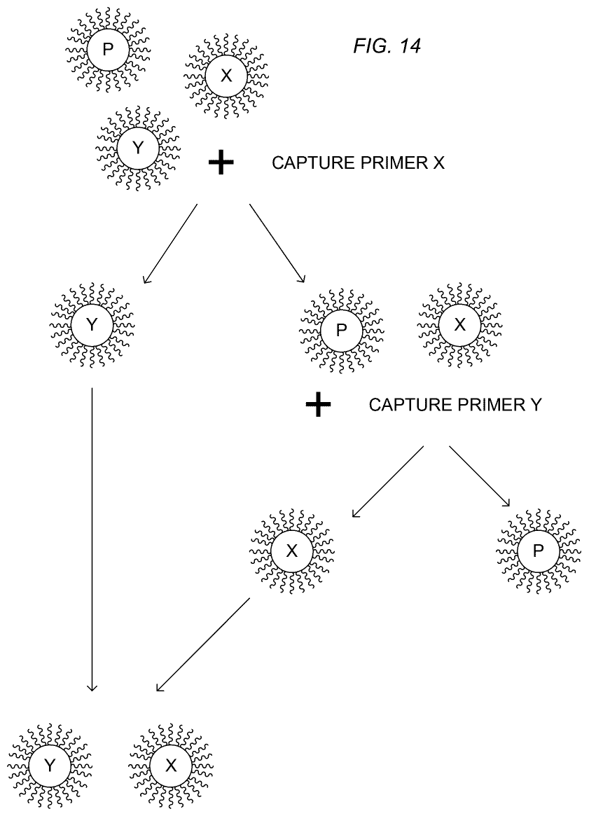

The process can be extended to systems that utilize more than two enrichment barcode sequences. For example, as illustrated in FIG. 13, three different enrichment barcode regions can be utilized. The resulting population of amplified nucleic acid supports can include a set of monoclonal supports having target nucleic acids having an enrichment region of the first sequence (X), a set of monoclonal supports having target nucleic acids within enrichment region of the second sequence (Y), and a set of monoclonal supports with target nucleic acids having an enrichment region of a third sequence (Z). The population of amplified nucleic acid supports can also include polyclonal supports having target nucleotides with different enrichment barcode regions of different sequences. For example, the population of amplified nucleic acid supports can include polyclonal supports having target nucleic acids with enrichment regions of the first sequence and enrichment regions of the second sequence (PXY), polyclonal supports adding target nucleic acids with enrichment regions of the first sequence and enrichment regions of the third sequence (PXZ), polyclonal supports having target nucleic acids with enrichment regions of the second sequence and target nucleic acids with enrichment regions of the third sequence (PYZ), or polyclonal supports with all three of the enrichment regions (not illustrate). The population can also include dud beads that do not include nucleic acids or dead supports (D).

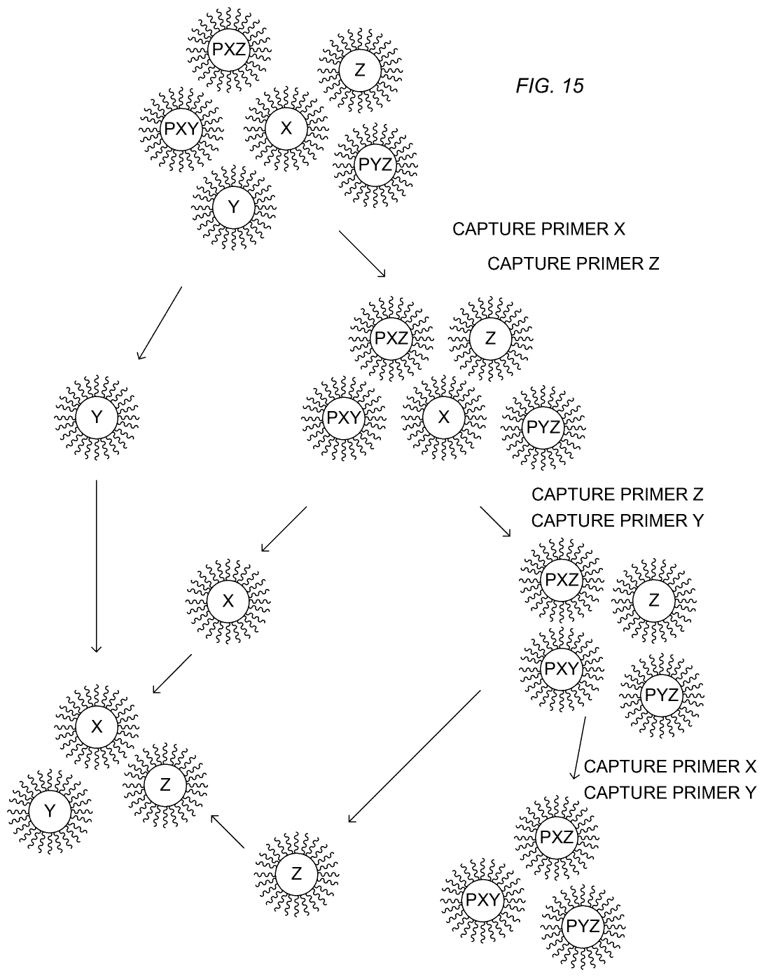

In an example, a process to separate polyclonal beads in which enrichment regions of two different sequences are present is illustrated in FIG. 14. An enrichment probe with a region complementary to the enrichment region of the first sequence (X) can be applied along with magnetic beads to the population. The enrichment probe can bind to target nucleic acids having an enrichment barcode region of the first sequence, and associated supports can bind to magnetic beads that bind to the enrichment probe. Magnetic separation can be performed.

Accordingly, monoclonal supports having target nucleic acids with an enrichment barcode region of the second sequence (Y) are separated from monoclonal supports having target nucleic acids with enrichment barcode region of the first sequence (X) and polyclonal supports having at least some target nucleic acids with enrichment region barcode region of the first sequence (P).

An enrichment probe with a region complementary to the enrichment barcode region of the second sequence (Y) can be applied to the dispersion with polyclonal nucleic acid supports. The enrichment probe binds to the polyclonal supports (P) and may not bind to supports that include target nucleic acids with enrichment barcode regions of the first sequence (X). The enrichment probes can bind to magnetic beads and magnetic separation can be performed. As such, monoclonal supports include target nucleic acids with enrichment barcode region of the first sequence (X) can be separated from polyclonal supports that include at least some target nucleic acids with an enrichment region of the second sequence (P). The sets of monoclonal supports can be combined resulting in an enriched population with a greater percentage of monoclonal supports.

In another example illustrated in FIG. 15, a method is illustrated that utilizes three different enrichment barcode region sequences. The method can be extended to utilize three or more different enrichment barcode region sequences.

Following amplification, the population of nucleic acid supports can include monoclonal supports that have target nucleic acids with enrichment regions of the one of the three enrichment region sequences (X, Y, or Z). The population of nucleic acid supports can further include polyclonal supports that include target nucleic acids with enrichment regions of one of the three enrichment barcode region sequences and target nucleic acids with enrichment regions of another of the three enrichment barcode region sequences (XY, XZ, YZ) or polyclonal supports with all species of the enrichment regions (not illustrated).

In a first separation step, an enrichment probe including a region complementary to a first enrichment barcode sequence (X) and an enrichment probe including a region complementary to a third enrichment barcode sequence (Z) can be used to isolate monoclonal supports with target nucleic acids having enrichment barcode regions of the second enrichment barcode region sequence (Y).

In a second separation step, an enrichment probe including a region complementary to the second enrichment barcode sequence (Y) and an enrichment probe including a region complementary to the third enrichment barcode sequence (Z) can be used. Monoclonal supports including target nucleic acids with enrichment barcode regions of the first enrichment barcode region sequence (X) can be isolated.

In a third separation step, an enrichment probe including a region complementary to the first enrichment barcode sequence (X) and an enrichment probe including a region complementary to the second enrichment barcode sequence (Y) can be used. Monoclonal supports having target nucleic acids with enrichment barcode regions of the third enrichment barcode sequence (Z) can be isolated.

The isolated monoclonal supports (X, Y, Z) can be combined to form a population of enriched monoclonal supports. The population of enriched monoclonal supports includes a higher concentration or higher percentage of monoclonal supports relative to polyclonal supports.

The above methods can be extended to systems including four or more types of the enrichment barcodes. In particular, the method can be extended to a system including four types of barcode sequences wherein each separation uses three capture primers with regions complementary to three types of enrichment barcode sequences.

### Amplification

Target nucleic acids are seeded onto supports, such as bead supports. For example, FIG. 10 illustrates an example method for seeding a target nucleic acid on a support. The seeded target nucleic acid can be amplified or templated—i.e., copies of the target nucleic acid are replicated on the bead support. Amplification can be performed prior to separating polyclonal supports. Alternatively, amplification can be performed after separating polyclonal supports.

In some embodiments, the methods for seeding template nucleic acid molecules onto one or more supports, as well as the templating reactions, typically use one or more enzymes capable of polymerization. In any of the embodiments of the present teachings, the one or more enzymes capable of polymerization include at least one polymerase. In some embodiments, the at least one polymerase includes a thermostable or thermolabile polymerase. In some embodiments, the at least one polymerase includes a biologically active fragment of a DNA or RNA polymerase that maintains sufficient catalytic activity to polymerize or incorporate at least one nucleotide under any suitable conditions. In various embodiments, the at least one polymerase includes a mutated DNA or RNA polymerase that maintains sufficient catalytic activity to perform nucleotide polymerization under any suitable conditions. In various embodiments, the at least one polymerase includes one or more amino acid mutations that maintains sufficient catalytic activity to perform polymerization. The polymerase optionally can have, or lack, exonuclease activity. In some embodiments, the polymerase has 5′ to 3′ exonuclease activity, 3′ to 5′ exonuclease activity, or both. Optionally, the polymerase lacks any one or more of such exonuclease activities. In some embodiments, the polymerase has strand-displacing activity. Examples of useful strand-displacing polymerases include Bacteriophage @29 DNA polymerase and Bst DNA polymerase.

In some embodiments, a polymerase includes any enzyme or fragment or subunit thereof, that can catalyze polymerization of nucleotides and/or nucleotide analogs. In some embodiments, a polymerase requires an extendible 3′ end. For example, a polymerase requires a terminal 3′ OH of a nucleic acid primer to initiate nucleotide polymerization. The polymerase can be other than a thermostable polymerase. For example, the polymerase can be active at 37° C. and/or more active at 37° C. than at 50° C., 60° C., 70° C. or higher. In various embodiments, the polymerase can be active and/or more active at 42° C., 45° C., 50° C., 55° C., or 60° C. than at 37° C.

A polymerase can include any enzyme that can catalyze the polymerization of nucleotides (including analogs thereof) into a nucleic acid strand. Typically, but not necessarily, such nucleotide polymerization can occur in a template-dependent fashion. In some embodiments, a polymerase is a high-fidelity polymerase. Such polymerases can include, without limitation, naturally-occurring polymerases and any subunits and truncations thereof, mutant polymerases, variant polymerases, recombinant, fusion or otherwise engineered polymerases, chemically-modified polymerases, synthetic molecules or assemblies, and any analogs, derivatives, or fragments thereof that retain the ability to catalyze such polymerization. Optionally, the polymerase is a mutant polymerase with one or more mutations involving the replacement of one or more amino acids with other amino acids, the insertion or deletion of one or more amino acids from the polymerase, or the linkage of parts of two or more polymerases. The term “polymerase” and its variants, as used herein, also refers to fusion proteins including at least two portions linked to each other, where the first portion can include a peptide that can catalyze the polymerization of nucleotides into a nucleic acid strand and is linked to a second portion that can include a second polypeptide, such as, for example, a reporter enzyme or a processivity-enhancing domain. Typically, the polymerase includes one or more active sites at which nucleotide binding and/or catalysis of nucleotide polymerization can occur. In some embodiments, a polymerase can include or lack other enzymatic activities, such as for example, 3′ to 5′ exonuclease activity or 5′ to 3′ exonuclease activity. In some embodiments, a polymerase is isolated from a cell or generated using recombinant DNA technology or chemical synthesis methods. In any of the embodiments of the present teachings, a polymerase is expressed in prokaryote, eukaryote, viral, or phage organisms. In various embodiments, a polymerase is a DNA polymerase and include without limitation bacterial DNA polymerases, eukaryotic DNA polymerases, archaeal DNA polymerases, viral DNA polymerases, and phage DNA polymerases. In various embodiments, the expressed polymerase is purified using methods known in the art. In some embodiments, a polymerase is post-translationally modified proteins or fragments thereof.

In some embodiments, the polymerase includes any one or more polymerases, or biologically active fragments of a polymerase, as described in U.S. Patent Publ. No. 2011/0262903 to Davidson et al., published Oct. 27, 2011, and/or International PCT Publ. No. WO 2013/023176 to Vander Horn et al., published Feb. 14, 2013, herein incorporated by reference in their entireties.

In some embodiments, a polymerase is a replicase, DNA-dependent polymerase, primases, RNA-dependent polymerase (including RNA-dependent DNA polymerases such as, for example, reverse transcriptases), a thermo-labile polymerase, or a thermostable polymerase. In some embodiments, a polymerase is any Family A or B type polymerase. Many types of Family A (e.g., E. coli Pol I), B (e.g., E. coli Pol II), C (e.g., E. coli Pol III), D (e.g., Euryarchaeotic Pol II), X (e.g., human Pol beta), and Y (e.g., E. coli UmuC/DinB and eukaryotic RAD30/xeroderma pigmentosum variants) polymerases are described in Rothwell and Watsman 2005 Advances in Protein Chemistry 71:401-440. In some embodiments, a polymerase is a T3, T5, T7, or SP6 RNA polymerase.

In some embodiments, nucleic acid amplification reactions are conducted with one type or a mixture of polymerases and/or ligases. In some embodiments, nucleic acid amplification reactions are conducted with a low-fidelity or high-fidelity polymerase or without regard to fidelity.

An exemplary polymerase is Bst DNA Polymerase (Exonuclease Minus), is a 67 kDa Bacillus stearothermophilus DNA Polymerase protein (large fragment), exemplified in accession number 2BDP_A, which has 5′ to 3′ polymerase activity and strand displacement activity but lacks 3′ to 5′ exonuclease activity. Other polymerases include Taq DNA polymerase I from Thermus aquaticus (exemplified by accession number 1TAQ), Eco DNA polymerase I from Escherichia coli (accession number P00582), Aea DNA polymerase I from Aquifex aeolicus (accession number 067779), or functional fragments or variants thereof, e.g., with at least 80%, 85%, 90%, 95% or 99% sequence identity at the nucleotide level.

In illustrative embodiments, the DNA polymerase is a Bsu DNA polymerase (large fragment (NEB)). Bsu DNA Polymerase I, Large Fragment retains 5′ to 3′ polymerase activity of the Bacillus subtilis DNA polymerase I (1), but lacks 5′ to 3′ exonuclease domain. In certain embodiments, the Bsu DNA Polymerase large fragment lacks 3′ to 5′ exonuclease activity. In various embodiments, Bsu DNA Polymerase large fragment has optimal activity at 37° C.

In certain illustrative embodiments, especially where the seeding reaction is an RPA reaction, the one or more enzymes capable of polymerization include a T5 or T7 DNA polymerase. In some embodiments, the one or more enzymes capable of polymerization include a T5 or T7 DNA polymerase having one or more amino acid mutations that reduce 3′ to 5′ exonuclease activity. In some embodiments, the T5 or T7 DNA polymerase having one or more amino acid mutations that reduce the 3 to 5′ exonuclease activity does not contain an amino acid mutation that disrupts the processivity of the T5 or T7 DNA polymerase. In some embodiments, the T5 or T7 DNA polymerase includes one or more amino acid mutations that eliminate detectable 3′ to 5′ exonuclease activity; and wherein the one or more amino acid mutations do not disrupt processivity of the T5 or T7 DNA polymerase. In certain illustrative embodiments, the seeding reaction mixture includes a Sau polymerase, T7 DNA polymerase with reduced 3′ to 5′ exonuclease activity, Bsu polymerase, or a combination thereof. These polymerases that are especially well suited for an RPA reaction, are well-suited not only for the seeding reaction, but the templating reaction as well.

In some embodiments, the one or more enzymes capable of polymerization include any suitable RNA polymerase. Suitable RNA polymerases include, without limitation, T3, T5, T7, and SP6 RNA polymerases.

In various embodiments, template nucleic acid molecules used in any of the methods of the present teachings, including the seeding reaction and the templating reaction, typically include a first primer binding sequence (“forward”) and optionally a second primer binding sequence (“reverse”). Accordingly, the seeding reaction mixture and the templating reaction include a population of first primers and optionally a population of second primers that bind the forward primer binding and reverse primer binding sequences, respectively. In some embodiments, the first and second primers are referred to as a primer pair. In some embodiments, the first primers or the second primers are typically universal primers. The first primer can bind to either the forward primer binding sequence or the reverse primer binding sequence and the second primer can bind to either the forward primer binding sequence or the reverse primer binding sequence.

In any of the disclosed embodiments, the reaction mixture includes a population of first primers and a population of second primers that bind to sequences within the template nucleic acid molecules. The population of first primers can be identical copies or different sequences. The population of second primers can be identical copies or different sequences. However, the population of first primers and optional second primers, are typically a universal primer and all copies are identical. Thus, in illustrative embodiments, the population of first primers and the population of second primers are both be universal primers that bind universal primer binding sequences on the template nucleic acid molecules. In other embodiments, both the population of first primers and the population of second primers are target-specific primers. The population of first primers and the population of second primers can have the same or different sequences. In any of embodiments of the present teachings, the population of first primers and/or the population of second primers are attached to one or more supports prior to incubation with the seeding reaction mixture. In other embodiments, the population of first primers and/or the population of second primers are in solution during incubation with the seeding reaction mixture. In illustrative embodiments, the population of first primers is attached to one or more supports prior to incubation with the seeding reaction mixture and the population of second primers is typically in solution during incubation with the seeding reaction mixture.

Thus, in these illustrative embodiments that include a population of immobilized first primers and a population of second primers in solution, not to be limited by theory, during the seeding reaction, a template nucleic acid is at least partially denatured, and the first primer binding site on the template binds to a first template attached to a solid support. The first primer is used by a polymerase to generate a complementary strand to one strand of the template nucleic acid. That complementary strand is now covalently attached to the solid support through the primer. A second primer in solution is in a complex with the recombinase and binds to a primer binding site on the complementary strand, thus partially denaturing the bound template nucleic acid molecule. A polymerase uses the primer to synthesize a new strand, identical to the original template nucleic acid strand. This strand is then believed to be partially denatured by the binding of the complex of a recombinase and a nearby first primer attached to the solid support, and the polymerase synthesizes another complementary strand. Through repeated steps of this process, a substantially monoclonal population of template nucleic acid molecules is generated during the seeding reaction, and further amplified during a templating reaction.

In some embodiments, the disclosure relates generally to methods, as well as systems, compositions, kits, and apparatuses, in which the primers typically have a free 3′ hydroxyl. In some embodiments, the primers are polymers of ribonucleotides, deoxyribonucleotides, or analogs thereof. In some embodiments, primers are naturally-occurring, synthetic, recombinant, cloned, amplified, or unamplified forms. In some embodiments, the primers include phosphodiester linkages between all nucleotides. In any of the embodiments of the present teachings, the primers are between about 5 and 100 nucleotides in length, for example between about 5 and 80 nucleotides in length, between about 5 and 60 nucleotides in length, between about 5 and 40 nucleotides in length, between about 10 and 75 nucleotides in length, between about 15 and 75 nucleotides in length, or between about 20 and 50 nucleotides in length.

In some embodiments, at least one of the primers has modifications. For example, ribonucleotide, deoxyribonucleotides, or analogs thereof can have biotin or azide attached to them. In some embodiments, the ribonucleotide, deoxyribonucleotides, or analogs thereof have attached fluorophores, phosphorylation, or spacers. In some embodiments, the primers are blocked and/or are fusion primers or fusion polynucleotides where different regions of the primers or polynucleotides are designed to bind to one of two primer binding sites and/or are designed to be bound by one of two primers.

In some embodiments, the primers are blocked primers to prevent extension from the 3′ end of the primer. In some embodiments, the blocked primers are tailed primers wherein 5′ end includes a sequence that is non-complementary to the template nucleic acid molecules. This 5′ sequence can be used as a template for primer extension reactions. In some embodiments, the primers are blocked wherein 5′ domain is 15 to 30 nucleotides in length. In some embodiments, the primers include a blocking moiety at their 5′ or 3′ end, or at 5′ and 3′ ends. In a reaction that involves primer extension (e.g., seeding amplification), the blocking moiety at 3′ end of the blocked fusion primers can reduce the level of primer-dimer formation. In certain embodiments, the seeding reaction mixture includes a blocked primer wherein 3′ domain is 14 to 25 nucleotides in length. In other embodiments, 3′ domain is 15 to 25 nucleotides in length. In still other embodiments, 5′ domain is at least 15 nucleotides and 3′ domain is at least 10 nucleotides, wherein the length of the primer does not exceed 100, 90, 80, 75, 70, 60, or 50 nucleotides. In embodiments, a 3′ nucleotide of 3′ domain of the forward primer is mismatched to the forward primer binding sequence. In embodiments, the ribobase separating 5′ domain and 3′ domain of the blocked primer includes rU, rG, rC, or rA. In certain embodiments, the ribobase separating 5′ domain and 3′ domain of the blocked primer includes rC. In any of the embodiments of the present teachings, 3′ domain of the blocked primers is 14 to 20 nucleotides in length and the ribobase is rU, rG, rC or rA.

In any of the embodiments of the present teachings, the reaction mixture includes an enzyme to remove a portion of the blocked primers to leave a free 3′ OH. After removing the blocked end of the primer, a polymerase can initiate replication from the free 3′ OH to begin replicating the template strand. In some embodiments, this enzyme is an RNase, especially RNase H. A skilled artisan will recognize other compositions of blocked primers to use and suitable enzymes for removing a portion of the blocked primers.

The seeding reaction mixture, as well an any other amplification reaction in the methods provided here, including the templating reaction mixture, typically includes a source of nucleotides, or analogs thereof, that is used by the polymerase as substrates for an extension reaction. In any of the embodiments of the present teachings, the seeding reaction mixture typically includes nucleotides (dNTPs) for strand extension of the template nucleic acid molecules resulting in a substantially monoclonal population of the template nucleic acid molecule sequence attached to one or more supports. In some embodiments, nucleotides are not extrinsically labeled. For example, the nucleotides can be naturally-occurring nucleotides or synthetic analogs that do not include fluorescent moieties, dyes, or other extrinsic optically detectable labels. Optionally, the nucleotides do not include groups that terminate nucleic acid synthesis (e.g., dideoxy groups, reversible terminators, and the like). In other embodiments, the nucleotides include a label or tag.

In some embodiments, methods for nucleic acid amplification includes at least one cofactor, for example a cofactor that enhances activity of a DNA or RNA polymerase. In some embodiments, a cofactor includes one or more divalent cations. Examples of divalent cations include magnesium, manganese, and calcium. In various embodiments, the seeding reaction mixture includes a buffer containing one or more divalent cation. In illustrative embodiments, the buffer contains magnesium or manganese ions. In any of the embodiments of the present teachings, the seeding reaction or the templating reaction is initiated by the addition of a cofactor, especially a divalent cation. In some embodiments, the seeding reaction mixture used herein for nucleic acid amplification may include at least one cofactor for recombinase assembly on nucleic acids or for homologous nucleic acid pairing. In some embodiments, a cofactor includes any form of ATP including ATP and ATP/S. In some embodiments, methods for nucleic acid amplification includes at least one cofactor that regenerates ATP. For example, a cofactor can include an enzyme system that converts ADP to ATP. In some embodiments, a cofactor enzyme system is phosphocreatine and creatine kinase.

In any aspects of the present teachings, the seeding reaction mixture includes components to partially denature template nucleic acid molecules. In some embodiments, partially denaturing conditions include treating or contacting the template nucleic acid molecules to be amplified with one or more enzymes that are capable of partially denaturing the nucleic acid template, optionally in a sequence-specific or sequence-directed manner, as in an RPA reaction. In some embodiments, at least one enzyme catalyzes strand invasion and/or unwinding, optionally in a sequence-specific manner. Optionally, the one or more enzymes include one or more enzymes selected from the following: recombinases, topoisomerases, and helicases. In some embodiments, partially denaturing the template includes contacting the template with a recombinase and forming a nucleoprotein complex including the recombinase. Optionally, the template nucleic acid molecule is contacted with a recombinase in the presence of a first and optionally a second primer. Partially denaturing can include catalyzing strand exchange using the recombinase and hybridizing the first primer to the first primer binding sequence (or hybridizing the second primer to the second primer binding sequence). In some embodiments, partially denaturing includes performing strand exchange and hybridizing both the first primer to the first primer binding sequence and the second primer to the second primer binding sequence using the recombinase.

In some embodiments, partially denaturing the template nucleic acid molecule includes contacting the template with one or more recombinases or nucleoprotein complexes. At least one of the nucleoprotein complexes can include a recombinase. At least one of the nucleoprotein complexes can include a primer (e.g., a first primer or a second primer, or a primer including a sequence complementary to a corresponding primer binding sequence in the template). In some embodiments, partially denaturing the template includes contacting the template with a nucleoprotein complex including a primer. Partially denaturing can include hybridizing the primer of the nucleoprotein complex to the corresponding primer binding sequence in the template, thereby forming a primer-template duplex. In some embodiments, partially denaturing the template nucleic acid molecule includes contacting the template with a first nucleoprotein complex including a first primer. Partially denaturing can include hybridizing the first primer of the first nucleoprotein complex to the first primer binding sequence of the forward strand, thereby forming a first primer-template duplex. In some embodiments, partially denaturing the template includes contacting the template with a second nucleoprotein complex including a second primer. Partially denaturing can include hybridizing the second primer of the second nucleoprotein complex to the second primer binding sequence of the reverse strand, thereby forming a second primer-template duplex.

Accordingly, the seeding reaction mixtures of the present disclosure, and a templating reaction of the present disclosure, include a recombinase and partial denaturation and/or amplification, including any one or more steps or methods described herein, can be achieved using a recombinase and optionally a recombinase accessory protein. The recombinase can include any agent that is capable of inducing, or increasing the frequency of occurrence, of a recombination event. A recombination event includes any event whereby two different polynucleotides strands are recombined with each other. Recombination can include homologous recombination. The recombinase optionally can associate with (e.g., bind) a first primer. In some embodiments, an enzyme that catalyzes homologous recombination can form a nucleoprotein complex by binding a single-stranded template nucleic acid molecule. In some embodiments, a homologous recombination enzyme, as part of a nucleoprotein complex, can bind a homologous portion of a double-stranded polynucleotide. In some embodiments, the homologous portion of the polynucleotide hybridizes to at least a portion of the first primer. In some embodiments, the homologous portion of the polynucleotide is partially or completely complementary to at least a portion of the first primer. Suitable recombinases include RecA and its prokaryotic or eukaryotic homologues, or functional fragments or variants thereof, optionally in combination with one or more single-strand binding proteins (SSBs). In certain embodiments, the recombinase optionally coats ssDNA to form a nucleoprotein filament strand which invades a double-stranded region of homology on a template.

In some embodiments, a homologous recombination enzyme catalyzes strand invasion by forming a nucleoprotein complex and binding to a homologous portion of a double-stranded polynucleotide to form a recombination intermediate having a triple-strand structure (D-loop formation) (U.S. Pat. No. 5,223,414 to Zarling, U.S. Pat. Nos. 5,273,881 and 5,670,316 both to Sena, and U.S. Pat. Nos. 7,270,981, 7,399,590, 7,435,561, 7,666,598, 7,763,427, 8,017,339, 8,030,000, 8,062,850, and 8,071,308, herein incorporated by reference in their entireties).

The recombinase of the reaction mixtures, compositions, and kits includes any suitable agent that can promote recombination between polynucleotide molecules. The recombinase can be an enzyme that catalyzes homologous recombination. For example, the reaction mixture can include a recombinase that includes, or is derived from, a bacterial, eukaryotic, or viral (e.g., phage) recombinase enzyme.

In any of the embodiments of the present teachings, a homologous recombination enzyme is wild-type, mutant, recombinant, fusion, or fragments thereof. In some embodiments, a homologous recombination enzyme includes an enzyme from any organism, including myoviridae (e.g., uvsX from bacteriophage T4, RB69, and the like) Escherichia coli (e.g., recA) or human (e.g., RAD51). In embodiments, the reaction mixture includes one or more recombinases selected from uvsX, RecA, RadA, RadB, Rad51, a homologue thereof, a functional analog thereof, or a combination thereof. In illustrative embodiments, the recombinase is uvsX. The UvsX protein can be present, for example, at 50-1000 ng/μl, 100-750 ng/μl, 200-600 ng/μl, or 250 to 500 ng/μl.

In some embodiments, methods, kits, and compositions for nucleic acid amplification includes one or more recombinase accessory proteins in the seeding reaction mixture. For example, an accessory protein can improve the activity of a recombinase enzyme. In some embodiments, an accessory protein can bind single strands of template nucleic acid molecules or can load a recombinase onto a template nucleic acid molecule. In some embodiments, an accessory protein is wild-type, mutant, recombinant, fusion, or fragments thereof. In some embodiments, accessory proteins can originate from any combination of the same or different species as the recombinase enzymes that are used to conduct a nucleic acid amplification reaction. Accessory proteins can originate from any bacteriophage including a myoviral phage. Examples of a myoviral phage include T4, T2, T6, Rb69, Aeh1, KVP40, Acinetobacter phage 133, Aeromonas phage 65, cyanophage P-SSM2, cyanophage PSSM4, cyanophage S-PM2, Rb14, Rb32, Aeromonas phage 25, Vibrio phage nt-1, phi-1, Rb16, Rb43, Phage 31, phage 44RR2.8t, Rb49, phage Rb3, and phage LZ2. Accessory proteins can originate from any bacterial species, including Escherichia coli, Sulfolobus (e.g., S. solfataricus) or Methanococcus (e.g., M. jannaschii). In some embodiments, methods for nucleic acid amplification can include single-stranded binding proteins. Single-stranded binding proteins include myoviral gp32 (e.g., T4 or RB69), Sso SSB from Sulfolobus solfataricus, MjA SSB from Methanococcus jannaschii, and E. coli SSB protein.

In some embodiments, methods for nucleic acid amplification include proteins that improve recombinase loading onto a nucleic acid. For example, UvsY protein is a recombinase-loading protein. In some embodiments, the reaction mixture includes recombinase accessory proteins. In illustrative embodiments, the recombinase accessory protein is uvsY. UvsY can be present between about 20 and 500 ng/μl, for example, between about 20 and 250 ng/μl, or between about 20 and 125 ng/μl. In a non-limiting example, UvsY is present at between 75 and 125 ng/μl.

In any of the embodiments of the present teachings, the seeding reaction is carried out under isothermal conditions. In some embodiments, isothermal conditions include a reaction subjected to a temperature variation which is constrained within a limited range during at least some portion of the amplification (or the entire amplification process), including for example a temperature variation that is equal or less than about 10° C., or about 5° C., or about 1-5° C., or about 0.1-1° C., or less than about 0.1° C., or, for example a temperature variation is equal or less than or 10° C., or 5° C., or 1-5° C., or 0.1-1° C., or less than 0.1° C. The temperature of the isothermal reaction can be typically between about 15° C. and 65° C., for example between about 15° C. and 55° C., between about 15° C. and 45° C., between about 15° C. and 37° C., between about 30° C. and 60° C., between about 40° C. and 60° C., between about 55° C. and 60° C., between about 35° C. and 45° C., or between about 37° C. and 42° C. In other embodiments, the seeding reaction is not exposed to a temperature above 40° C., 41° C., 42° C., 43° C., 45° C., or 50° C. Accordingly, in certain embodiments, the reaction mixture is not exposed to hot start conditions. However, it is understood the enzymes used at these temperatures will need to be optimized in combination and may require changes in the enzyme, for example, using a different DNA polymerase, such as Bst instead of Bsu. A rate limiting enzyme may be the polymerase, wherein a high concentration or excess (i.e., non-limiting) amount of the polymerase or a lower temperature ensures the amplification reaction proceeds based on the kinetics of the polymerase.

The seeding reaction can be performed for 0.25 minutes to 240 minutes, thereby amplifying the nucleic acid template. In certain embodiments, the seeding reaction is performed for between about 0.25 and 240 minutes, for example between about 0.25 and 120 minutes, between about 0.25 and 60 minutes, between about 0.25 and 30 minutes, between about 0.25 and 15 minutes between about 0.25 and 10 minutes, between about 0.25 and 7.5 minutes, between about 0.25 and 5 minutes, or between about 2 and 5 minutes. In further illustrative embodiments, the seeding reaction is performed for between about 1.5 and 10 minutes, for example between about 1.5 and 8 minutes, between about 1.5 and 6 minutes, between about 1.5 and 5 minutes, or between about 1.5 and 4 minutes, the reaction is isothermal, and the temperature of the reaction is between about 35° C. and 65° C., for example between about 35° C. and 55° C., between about 35° C. and 45° C., between about 30° C. and 60° C., between about 40° C. and 60° C., between about 40° C. and 55° C., between about 50° C. and 60° C., or between about 37° C. and 42° C. For example, the seeding reaction can be between performed for between about 1 and 10 minutes in an isothermal reaction where the temperature of the reaction can be between about 35° C. and 45° C. or the seeding reaction can be between performed for between about 2 and 5 minutes in an isothermal reaction where the temperature of the reaction can be between about 37° C. and 42° C.

The seeding reaction result in the formation of a population of seeded supports with populations of substantially monoclonal template nucleic acid molecules attached thereto. In various aspects, the substantially monoclonal populations of template nucleic acid molecules are template nucleic acid molecules with at least 50%, 55%, 60%, 65%, 70%, 75%, 80%, 85%, 90%, 95%, 96%, 97%, 98%, 99%, 99.5%, 99.9% or 100% identity at the sequence level. In some embodiments, the percentage of substantially monoclonal template nucleic acid molecules attached to a seeded support are 50%, 55%, 60%, 65%, 70%, 75%, 80%, 85%, 90%, or 95% of the template nucleic acid molecules attached thereto.

In various embodiments, at least 5%, 10%, 15%, 20%, 25%, 30%, 35%, 40%, 45% 50%, 55%, 60%, 65%, 70%, 75%, 80%, 85%, 90%, 95%, 96%, 97%, 98%, or 99% of the supports in a population of seeded supports have substantially monoclonal populations of nucleic acid molecules attached during the seeding reaction. In illustrative embodiments, at least 50%, 55%, 60%, 65%, 70%, 75%, 80%, 85%, 90%, 95%, 96%, 97%, 98%, or 99% of the supports in a population of seeded supports have substantially monoclonal populations of nucleic acid molecules attached during the seeding reaction.

In some embodiments, a seeding reaction generate supports having zero template nucleic acid molecules attached thereto (empty solid supports), other seeded supports having one type of template nucleic acid molecule attached thereto, and other seeded supports having more than one type of template nucleic acid molecule attached thereto. In any of the embodiments of the present teachings, the number of attached template nucleic acid molecules in the populations of substantially monoclonal template nucleic acid molecules attached to one or more seeded supports are the seeding number. In any of the disclosed embodiments, the seeding number is between about 1 and 150,000 template nucleic acid molecules, for example between about 1 and 100,000, between about 1 and 75,000, between about 1 and 50,000, between about 1 and 25,000, between about 1 and 10,000, between about 1 and 5,000, or between about 1 and 2,500 template nucleic acid molecules. In illustrative embodiments, the seeding number is between about 10 and 100,000 template nucleic acid molecules, for example between about 10 and 75,000, between about 10 and 50,000, between about 10 and 25,000, between about 10 and 10,000, between about 10 and 5,000, or between about 10 and 2,500 template nucleic acid molecules.

In some embodiments, after the seeding reaction, a majority of any primers attached to a support are not bound to a template nucleic acid molecule. These unbound primers can be used in the subsequent templating reaction for further amplification of the template nucleic acid molecules. For example, after the seeding reaction, at least 90%, 95%, 96%, 97%, 98%, or 99% of primers attached to a support are typically not bound to a template nucleic acid molecule.

In some embodiments, the disclosure relates generally to methods, as well as systems, compositions, kits, and apparatuses, wherein the methods typically include a templating reaction after the seeding reaction wherein the template nucleic acid molecules on the seeded supports are further amplified (herein referred to as the templating reaction). The templating reaction mixture does not include additional template nucleic acid molecules in solution such that the template nucleic acid molecules attached to the one or more seeded supports are the predominant or only source of template nucleic acid molecules in the templating reaction mixture before the templating reaction is initiated. In illustrative embodiments, one or more washes are carried out on the one or more seeded supports before introducing them into the templating reaction mixture. In some embodiments, less than 10%, 9%, 8%, 7%, 6%, 5%, 4%, 3%, 2%, 1%, or 0.1% of the template nucleic acid molecules in solution at the end of the seeding reaction mixture are present in the templating reaction. In any of the embodiments of the present teachings, the percentage of template nucleic acid molecules in the templating reaction mixture that are attached (have been seeded) on one or more supports before the templating reaction is initiated are at least 50%, 55%, 60%, 65%, 70%, 75%, 80%, 85%, 90%, 95%, 96%, 97%, 98%, 99%, 99.5%, or 99.9% of the template nucleic acid molecules in the reaction mixture.

In any of the embodiments of the present teachings, the templating reaction is typically an RPA reaction. As such, any details regarding components and conditions for an RPA reaction discussed above for a seeding reaction apply to the templating reaction except that template nucleic acids are typically present in the initial reaction mixture for a seeding reaction, but not for a templating reaction. The templating reaction mixture typically includes all or some of the following: one or more seeded solid supports that include a population of attached substantially identical first primers and have substantially monoclonal template nucleic acid molecules attached thereto, a polymerase, a recombinase, an optional single-stranded binding protein, an optional recombinase loading protein, an optional second or reverse primer, which can be attached to the solid support, but in illustrative examples, is in solution, dNTPs, ATP, a buffer, and optionally one or both of phosphocreatine and creatine kinase. A divalent cation, such as MgCl2 or Mg(OAc)2, can be added to start the reaction. In various embodiments, the buffer included a crowding agent, such as PEG, Tris buffer, and/or a potassium acetate salt. A forward primer binding sequence on template nucleic acid molecules is complementary or identical to at least a portion of the forward primer and the optional reverse primer binding sequence on the template nucleic acid molecules is complementary or identical to at least a portion of the reverse primer. Reaction mixtures for templating reactions themselves form separate aspects of the invention. In further illustrative embodiments, the RPA templating reaction is a bulk isothermal amplification or is performed in wells of a multi-well solid support (e.g., see the seeding amplification reaction discussed further herein).

Optionally, the amplification or templating reaction can be performed in two steps. For each step, a primer complementary to the z-primer region can be used as the second primer. Alternatively, a primer complementary to the z-primer region can be used in the first step and a primer complementary to the a-primer region can be used in the second step. Monoclonal enrichment can be performed before the amplification/templating reaction, following the first step and before the second step, or following the second step. For two step amplification/templating see, for example, WO 2020/227031.

### Enrichment

In an example, bead supports are separated based on the nature of the enrichment barcode, for example, as described in relation to FIG. 8. A bead support including a target polynucleotide to be sequenced is attached to a magnetic bead to form a bead assembly, for example, using an enrichment probe that binds to moieties on the magnetic bead. In some embodiments, the magnetic bead is attached to the bead support by a double stranded polynucleotide linkage. In some instances, a linker moiety is hybridized to a portion of the target polynucleotide on the bead support. In an example, the linker moiety attaches to a complementary linker moiety on the magnetic bead. In another example, the template polynucleotide used to form the target nucleic acid attached to beads includes a linker moiety that attaches to the magnetic bead. In another example, the template polynucleotide complementary to target polynucleotide attached to the bead support is generated from a primer that is modified with a linker that attaches to the magnetic bead. The linker moiety attached to the polynucleotide and the linker moiety attached to the magnetic bead are, in some embodiments, complementary to and attach to each other. In an example, the linker moieties have affinity and include: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; or an oligonucleotide or polynucleotide and its corresponding complement. In a particular example, the linker moiety attached to the polynucleotide includes biotin and the linker moiety attached to the magnetic bead includes streptavidin.

For example, amplified bead supports are enriched using MyOne beads (ThermoFisher Scientific, Waltham, MA). A capture probe having a region complementary to one of the two or more enrichment barcode regions and having a capture moiety are added to the amplified bead supports and incubated. MyOne beads having a capture moiety complementary to the capture moiety of the capture probe are added to dispersions including amplified bead supports. The dispersion rotated for 15 minutes at room temperature, spun, and placed on a magnetic tube rack. After the beads are fully pelleted, the supernatant is removed and retained—the supernatant likely contains monoclonal supports having an enrichment barcode region not complementary to the capture probe. The sample is mixed with a 3 mM SDS solution. The sample are vortexed thoroughly, briefly spun, and placed on a magnetic tube rack for 2 minutes. The supernatant is removed, and the samples are removed from the magnetic tube rack. The beads are resuspended with a melt-off solution (e.g., 125 mM NaOH and 0.1% TWEEN® 20), vortexed thoroughly, and briefly spun. After a room temperature incubation, the bead solution is placed on a magnetic tube rack and the supernatant is transferred to a new tube. The tube is spun at maximum speed, and the supernatant is removed. Water is added to the templated bead supports. The tube is spun, and the supernatant is removed to leave a solution that may include polyclonal bead supports and monoclonal bead supports that have target nucleic acids with an enrichment barcode region complementary to the capture probe. The process can be repeated using a capture probe having a region complementary to a different one of the two or more enrichment barcode regions.

### Sequencing

In any of the disclosed embodiments, the amplified template nucleic acid molecules on the supports are sequenced. Sequencing methods can include any suitable method of sequencing known in the art. In some embodiments, template nucleic acid molecules that have been amplified according to the present teachings are used in any nucleic acid sequencing workflow, including sequencing by oligonucleotide probe ligation and detection (e.g., SOLID™ from Life Technologies, WO 2006/084131), probe-anchor ligation sequencing (e.g., Complete Genomics™ or Polonator™), sequencing-by-synthesis (e.g., Genetic Analyzer and HiSeq™, from Illumina), pyrophosphate sequencing (e.g., Genome Sequencer FLX from 454 Life Sciences), ion-sensitive sequencing (e.g., Personal Genome Machine (PGM™), Ion Proton™ Sequencer, Ion S5, Ion S5 XL, or Genexus™ all from Ion Torrent™ Systems, Inc.), single molecule sequencing platforms (e.g., HeliScope™ from Helicos™), nanopore sequencing via read of individual bases as they pass through the nanopores (e.g. MinION from Oxford Nanopore Technologies), chemical degradation sequencing, capillary electrophoresis, gel electrophoresis, and any other next-generation, massively parallel sequencing platforms.

### EXAMPLES

### Example 1

Genomic DNA from two samples is seeded onto beads and amplified in a bulk isothermal reaction.

### Generating Template Nucleic Acid Molecules

For example, sample DNA is amplified from genomic DNA using PCR primers 1, 12, and 13 from the Oncomine Focus Assay (OFA) (Thermo Fisher Scientific, Waltham, MA). Adapters that facilitated binding to immobilized primer B and a primer A and Enrichment Region in solution used in the seeding reaction, were added to the amplicons during 10 cycles of a second tailed PCR. The amplification generated 3 templates. The DNA (target nucleic acid) from the samples is combined in a single solution. The DNA from each sample has a differently sequenced enrichment barcode region.

### Seeding Reaction

Dilutions of the target nucleic acids are made and used to generate separate bead supports in a seeding reaction. The seeding reaction included 6.67 μl P1 ISPs with immobilized primer B (400,000,000 ISPs), 88.33 μl 1× Platinum HiFi Mix (Thermo Fisher Scientific, Waltham, MA), and 5 μl of the appropriate template dilution to generate a desired number of substantially monoclonal template molecules. The reaction mixture is placed in a thermocycler for a denaturation step of 98° C. for 2 min followed by 98° C. for 25 sec and 56° C. for 10 min to generate seeded bead supports.

The seeded P1 ISPs are washed twice in Ion OneTouch Wash Solution wash buffer in a total volume of 1 ml. After the first wash, the samples are centrifuged at >21,000 g for 8 min and the supernatant is removed to leave ˜100 μl. After a second wash and centrifugation, the supernatant is removed to leave ˜50 μl sample in the tube. The sample is then treated with 300 μl of freshly prepared melt off solution (125 mM NaOH, 0.1% TWEEN® 20) and thoroughly vortexed before incubation for 5 minutes at room temperature. These samples are then washed three times with nuclease-free (NF) H₂O in a final volume of 1 ml. After each wash, the samples are centrifuged at >21,000 g for 8 min and the supernatants are removed to leave ˜100 μl.

### Templating Reaction

The seeded P1 ISPs are combined with a primer mix including a primer having a sequence complementary to at least the Z-Primer in solution not attached to any substrate, wherein the template nucleic acid molecules include a Z-Primer binding site at or near the terminus opposite the proximal segment). Two Ion PGM™ Template IA (isothermal amplification) pellets were rehydrated, each with 720 μl Rehydration Buffer, vortexed, and briefly spun. Dehydrated Template IA pellets contained T7 polymerase, uvsX recombinase, uvsY recombinase loading protein, gp32 protein, Bsu DNA polymerase, dNTPs, ATP, thioredoxin, phosphocreatine, and creatine kinase. Four pellets from a TwistAmp™ Basic kit were rehydrated in 120 μL of Rehydration Buffer (25 mM Tris, pH 8.3; 5 mM DTT; 3 mM dNTP; 3.5625% Trehalose; 0.1 mg/ml Creatine Kinase; 1.1375 mg/ml Twist gp32; 0.4 mg/ml UvsX; 0.1 mg/ml UvsY; 0.25 mU/ul PPiase; 0.02 mg/ml Sau Pol; 0.03063 mg/ml T7 Dbl Exo-Pol; 0.0225 mg/ml Thioredoxin (5×); 1.425% PEG 35) supplied from the kit. The recombinase solution was vortexed and spun, then iced. To the seeded P1 ISPs, 360 μl of the rehydrated pellet solution were add. The solution is thoroughly vortexed and briefly spun. The templating reaction is performed at 40° C. for 30 minutes. To stop the templating reaction, 650 ul of 100 mM EDTA is added, the solution is vortexed, and briefly spun.

### Enriching the Templated ISPs

The templated ISPs are enriched using MyOne beads (ThermoFisher Scientific, Waltham, MA). Briefly, the stopped templating reaction, capture probes complementary to at least one enrichment barcode region, and 100 μl of MyOne beads are added to a tube. The tube is rotated for 15 minutes at room temperature, spun, and placed on a magnetic tube rack. After the beads were fully pelleted, the supernatant is removed and retained. The retained solution likely contains monoclonal beads having templated nucleic acids with enrichment barcode regions not complementary to the region of the capture probe. 500 μl 3 mM SDS solution is added to the tube. The tube is vortexed thoroughly, briefly spun, and placed on a magnetic tube rack for 2 minutes. The supernatant is removed, and the tube is removed from the magnetic tube rack. The beads are resuspended with 200 μl of melt-off solution (125 mM NaOH and 0.1% Tween 20), vortexed thoroughly, and briefly spun. After a 2 minute room temperature incubation, the bead solution is placed on a magnetic tube rack for 2 minutes and the supernatant is transferred to a new 0.2 ml tube. The tube is spun for 8 minutes at maximum speed and the supernatant is removed to leave ˜10-15 μl solution. 90 μl water is added to the templated ISPs and 2 μl is removed for analysis on a Guava easyCyte Flow Cytometer. The tube is spun for 5 minutes at 15,000 rcf and the supernatant is removed to leave 10 μl of solution containing the templated ISPs. The process can be repeated with different capture probes, and the resulting solutions of monoclonal bead supports combined.

### Sequencing

To the monoclonal supports, 20 μl of 100% PBST and 20 μl of sequencing primer are added to the ISPs and the tubes are vortexed and spun briefly. The sequencing primer is annealed according to the manufacturer's instructions using the Ion Torrent PI™ Sequencing HiQ 200 Kit (Life Technologies, Carlsbad, CA).

A standard sequencing reaction is conducted on an Ion Torrent PGM according to the manufacturer's instructions using an Ion Torrent PI™ Sequencing HiQ 200 Kit (Life Technologies, Carlsbad, CA). The sequencing signals are analyzed by Torrent Suite Software to determine the sequence present within the amplicon of these ISPs.

### Results

The bead supports enriched as described above generated better sequencing metrics than similar reactions without monoclonal enrichment. For example, the total number of usable reads increased, the percentage of usable reads increase, and coverage increased.

### Example 2

### Library Conversion:

Make library conversion mix in PCR tube or plate well. Setup at room temperature (or 25 C on instrument). Mix the following components in a total of 45 uL volume.
Library: 20 uL of Library+10 uL of Nuclease-Free water. Bring up volume with NF-water (Library+NF water=up to 30 uL).
Primers: 6 uL (3 uL each)
   trP1-AV4 fusion (3 uM): 3 uL
   Fus. 1 (3 uM): 3 uL
5×HiFi: 9 uL

Place tube on SimpliAmp and start the TC. Make sure the TC enters the 25 C initial priming stage. Skip the step to start PCR. Bring up the volume of the converted library to 100 uL with Nuclease-free water.

Add 90 uL of AMPure XP beads. Mix well by pipetting up and down, and vortex mixer at least 10 times. Incubate for 5 minutes at room temperature. Place the tube on an appropriate magnetic stand to separate beads from supernatant. After the solution is clear (about 3 minutes), carefully remove and discard the supernatant without disturbing the beads.

Add 200 uL of freshly prepared 80% ethanol to the tube while in the magnetic stand. Incubate at room temperature for 30 seconds, then carefully remove and discard the supernatant. Repeat one time. Completely remove the residual ethanol, and air-dry beads for 1 minute while the tube is on the magnetic stand with lid open.

Elute the target DNA from the beads with 11 uL of nuclease-free water (power-wash). Incubate the tube at room temperature for 2 minutes. Place the back on the magnet stand until the solution is clear. Transfer 10 uL of the supernatant to a clean PCR tube.

### Mix Reagents in 2 mL Tube:

3 uL of converted library ampured (CLA)
1800M AV4 (630k primvoler load) per Reaction==22.5 uL
8 uL of Amplification Primer Mix S v2.0
H20 up to 150 uL total volume==116.5 uL

Vortex and pop-spin reagent mixture. Add 720 uL of Rehydration Buffer to pellet. Add 750 uL (720 uL+30 uL) of Rehydrated pellet to 2 mL tube. Add 300 uL of Start Solution to 2 mL tube. Incubate reaction at 40 C for 30 minutes. After 30 minutes, put half of the reaction (600 uL) into a second 2 mL tube with 650 uL of 100 mM EDTA. Add 650 uL of 100 mM EDTA to the original reaction tube. Vortex both tubes thoroughly and popspin.

### Direct Enrichment/Clean-Up of ISPs

Add 100 uL of MyOnes (C1), to each tube, vortex 30 seconds, and incubate at room temperature for at least 15 minutes. Pop-spin tubes, and place on appropriate magnetic stand to separate beads (pellet) and remove supernatant. Remove from magnetic stand and add 1 mL of ion “Wash solution” to each tube, vortex for 30 seconds, pellet and remove supernatant-Perform this Step 2 times (2 ion washes).

Add 100 uL of Melt Off (100 mM NaOH) to each tube, vortex for 30 seconds, and incubate at room temperature for 5 minutes. Pellet Magnetic beads and combine the supernatants with ISPs into 0.2 mL tube. Centrifuge Spin at 15000 rpm for 10 minutes.

Carefully remove ˜90% of liquid, down to ˜20 uL, without disturbing the ISP pellet at the bottom of the tube. Perform water washes with Centrifuge Spins-Perform this Step 2 times. After final spin, resuspend ISP pellet in ˜20 uL of H2O by pipetting up and down, and vortexing.

### Monoclonal Enrichment

Start with 50 uL of cleaned ISPs from BulkIA Hybridize Capturing Primer to ISPs and Primer Dimer Removal. Add 20 uL of primer mix. Add 10 uL of 10× Binding Buffer. Add 20 uL of NF H2O. FastHyb. Wash MyOnes. Add 100 uL of MyOnes to mixture. Wash MyOnes-Repeat steps two times.

Add 200 uL of 1× Binding Buffer and resuspend pellet, vortex 30 sec. Pellet MyOnes and remove supernatant. Resuspend MyOnes with 100 uL of ISP/Primer Mix (from FastHyb). Incubate for 15 min. Recovery Bound ISPs from MyOnes. Pellet MyOnes in Magnet. Collect ˜100 uL Supernatant.

MeltOff ISPs. Thermal MeltOff-50 uL of Nuclease-free H2O. Incubate at 100 C for 5 min on PCR Block. Pellet MyOnes and collect supernatant.

5 uM of 99% non-biotin target Barcode: 1% Biotin-non-target Barcode(s). Incubate at room temperature for 5 min. Pellet MyOnes in Magnet. Collect supernatant, ˜100 uL, and set aside for late-Monoclonal MCE1 ISPs.

MeltOff ISPs. Thermal MeltOff. Add 50 uL of Nuclease-free H2O. Incubate at 100 C for 5 minutes (PCR Block). Pellet MyOnes and collect supernatants in new PCR tube-Monoclonal MCE2 and polyclonal ISPs. Repeat Steps 2-4 for MCE2.

### Load and Sequence Chip

Hybridize sequencing primer to ISPs in 40 uL Split ISPs into two tubes. Calculate the volume of ISP solution (uL) you want to load based on the total # of ISP you want to load.

Add 15 uL of Sequencing Primer. Add 15 uL 1× Annealing Buffer. Run FastHyb protocol.

| Stage 1 | Stage 2 | Stage 3 |
|---|---|---|
| 98 C. 2 min | 37 C. 2 min | 25 C. |

Wash Chip. Inject 200 uL of H2O into the chip-Perform Step two times. Inject 200 uL of IPA into the chip and vacuum dry. Spin Load ISPs into chip-Repeat the following steps with both split ISP pools. Add 10 uL of P1v3 buffer to a tube with 40 uL ISP pool.

Inject sample into chip and spin into chip for 2 min. Inject 1×800 uL Foam Scrape with 1×AB (**Foam AB). Inject 200 uL of 70% IPA/30% AB flush and vacuum dry-Perform Step two times. Inject 200 uL 1×AB and vacuum dry.

Repeat Steps with the second pool of ISPs.

Hybridize Sequencing Primer on chip.

Prepare 100 uL of sequencing primer mix (50 uL Sequencing primer+50 uL 1×AB). Inject Sequencing Primer Mix into chip and fill ports with remaining volume. Put chip on PCR machine and run Primerhyb Routine

| Stage 1 | Stage 2 | Stage 3 |
|---|---|---|
| 50 C. 2 min | 20 C. 2 min | 25 C. |

Inject 200 uL 1×AB into chip and vacuum dry. Bind PSP4 on chip. Prepare 55 uL of PSP4 polymerase mix (50 uL 1×AB+5 uL PSP4). Inject into flow-cell and incubate for 5 min at room temperature. Vacuum dry chip and then fill flow-cell with 50 uL of 1×AB.

### Sequence

### Example 3

### Capture Oligos:

MCE1:
(SEQ. #1)
TCAGTCTAGTCGTCACTACTGAGAGC
MCE2:
(SEQ. #2)
TCAGTACAGAGCACTCTACGTCATGC
MCE3:
(SEQ. #3)
TCAGAGTACAGGTCACTACTGAGAGC
MCE4:
(SEQ. #4)
TCAGACCTATCGTCACATCTGTGTGC

### Fusion Primers:

Fus1:
(SEQ. #5)
GTAGCATGGTGCCCGAGATGTCAGTCTAGTCGTCACTACTGAGAGCCCA
TCTCATCCCTGCGTGTC
Fus2:
(SEQ. #6)
GTAGCATGGTGCCCGAGATGTCAGTACAGAGCACTCTACGTCATGCCCA
TCTCATCCCTGCGTGTC
Fus3:
(SEQ. #7)
GTAGCATGGTGCCCGAGATGTCAGAGTACAGGTCACTACTGAGAGCCCA
TCTCATCCCTGCGTGTC
Fus4:
(SEQ. #8)
GTAGCATGGTGCCCGAGATGTCAGACCTATCGTCACATCTGTGTGCCCA
TCTCATCCCTGCGTGTC

### Amplification Primers:

Z:
(SEQ. #9)
GTAGCATGGTGCCCGAGATG
5 + Z:
(SEQ. #10)
ACGATGTAGCATGGTGCCCGAGATG
25 + Z-1:
(SEQ. #11)
CCCTGTCCATAAGGTCAGTAACGATGTAGCATGGTGCCCGAGAT

### Amplification Mix S v2:

| | | |
|---|---|---|
| 5 + Z | 100 μL of 100 μM | (final conc. 83 μM) |
| 25 + Z − 1 | 10 μL of 100 μM | (final conc. 8.3 μM) |
| 25 + Z − 1 − Bio | 10 μL of 100 μM | (final conc. 8.3 μM) |

### Amplification Mix L v2:

| | | |
|---|---|---|
| Z | 92 μL of 100 μM | (final conc. 92 μM) |
| 25 + Z − 1 − Bio | 8 μL of 100 μM | (final conc. 8 μM) |

Starting from BulkIA after ISPs are washed twice in nuclease-free water and pooled.

### Pre-Hybridization ISP Washes

Carefully remove all but ˜50 μL of supernatant without disturbing the ISPs and discard. Add 150 μL of 1% SDS (diluted in water) and vortex thoroughly. Spin for 10 minutes @ 15,000 rpm (max speed). Carefully remove all but ˜50 μL of supernatant without disturbing the ISPs and discard. Add 150 μL of 100 mM NaOH (diluted in water) and vortex thoroughly. Spin for 10 minutes @ 15,000 rpm (max speed).

Perform two washes of 150 uL nuclease-free water in the manner above, vortex ISPs in water and spin for 10 minutes @ 15,000 rpm each time. Resuspend the ISPs in ˜150 uL nuclease-free water and count. Resuspend MyOnes in MCE binding buffer. For each reaction take 200 μL of MyOnes into a tube and pellet them on a magnetic stand.

Remove and discard the supernatant. Resuspend the MyOnes in 200 μL of 1X Binding Buffer.

Hybridize capture primer to ISPs.

Prepare 40 μL of a 99:1 primer mix for each desired capture. Example for a 4 primer capture system:

| | Clonal BC1 | Clonal BC2 | Clonal BC3 | Clonal BC4 |
|---|---|---|---|---|
| | Mix | Mix | Mix | Mix |
| Non-bio | 20 μL of | 20 μL of | 20 μL of | 20 μL of |
| primer for | MCE1 | MCE2 | MCE3 | MCE4 |
| clonal | | | | |
| supernatant | | | | |
| Biotinylated | 6.7 μL each of | 6.7 μL each of | 6.7 μL each of | 6.7 μL each of |
| primers for | bio-MCE2, 3, 4 | bio-MCE1, 3, 4 | bio-MCE1, 2, 4 | bio-MCE1, 2, 3 |
| polyclonal | | | | |
| capture on | | | | |
| bead (1 μM) | | | | |

### Prepare

Add 50 μL containing 1 billion ISPs (add water if needed). Add 40 μL of 50 uM Primer Mix. Add 10 μL of 10× Binding Buffer.

Fasthyb (98 for 2 minutes' 37 C for 2 minutes) on PCR Bloc. Add 100 μl Mix to Pelleted MyOne. Pellet 100 μL of MyOnes in a PCR tube on a magnet and remove supernatant.

Wash MyOnes 2x. Resuspend in 200 ul of 1× Binding Buffer. Pellet and remove supernatant. Resuspend Pelleted MyOnes with 100 ul of ISP/Primer Mix. Transfer the full 100 ul of ISP Mix to the tube containing the MyOne pellet. Popspin “empty” tube and transfer the residual liquid.

Pipet mix thoroughly to ensure pellet is completely broken up. Vortex for about 10 seconds to ensure thorough mixing. Popspin tube to collect all solution back to bottom of tube. Incubate for 5 minutes.

Recover Bound ISPs from MyOnes. Pellet MyOne-ISP mixture on Magnet. Collect ˜100 ul Supernatant containing unbound ISPs and set aside for later.

MeltOff MyOne-Bound ISPs. Add 50 μl of MeltOff Solution to MyOne Pellet. Thoroughly Pipet mix to break-up the pellet.

Vortex for about 10 seconds to ensure thorough mixing. Popsin tube to collect all solution back to bottom of tube. Incubate for 5 minutes to dissociate ISPs from MyOnes. Pellet MyOnes On a Magnet and Collect Supernatant.

Repeat above steps with each Primer Mixture:
Clonal BC1 Pool in Binding Supernatant (keep),
Clonal BC2 Pool in Binding Supernatant (keep),
Primer Dimer Pool in Binding Supernatant (Throw away), and
Polyclonal Pool in MeltOff Supernatant (Throw away).

Prepare ISPs for loading. Pool Desired ISPs together (i.e. Clonal BC1+Clonal BC2) into a PCR tube. Spin Clean ISPs-Repeat 2x: Bring volume up to ˜200 ul with H20. Spin at maximum speed on the centrifuge for 10 minutes. Remove 120-150 ul of Supernatant (Being careful not to disturb the pellet). Bring volume up to desired final volume to 20 or 40 ul (for 1 or 2 shot loading). Vigorously pipet mix to resuspend the ISPs pellet in H20. Vortex for about 10 seconds to ensure thorough mixing.

Proceed with SOP Chip Loading.

In a first aspect of the disclosure, a method for enriching clonal populations is provided. The method includes preparing a population of target nucleic acids, each target nucleic acid of the population including a first segment at the 5′ end, a second segment at the 3′ end, and a third segment disposed between the first and second segments. The second segment includes a first region, a second region, and a third region, wherein the first segment is the same for each target nucleic acid of the population, the first and third regions of the second segment are the same for each target nucleic acid of the population, and the second region of the second segment has a sequence of one type of two or more types of sequences. The method further includes exposing the population of target nucleic acids to a plurality of supports, each support having a set of bound oligonucleotides the same as or complementary to the first segment, such that the target nucleic acids or their complements bind to the supports. The bound target nucleic acids are amplified in the presence of a primer at least complementary to the third region of the second segment to form supports including a plurality of copies of the target nucleic acids. This results in a first set of supports including monoclonal copies of a target nucleic acid with a second region having a sequence of a first type, a second set of supports including monoclonal copies of a target nucleic acid with a second region having a sequence of a second type, and a third set of supports including a polyclonal mixed set of copies of target nucleic acids with second regions having sequences of two or more types. The method also includes applying to the supports a capture primer having a sequence complementary to the third region and the second type of sequence, the capture primer including a binder moiety, applying a magnetic bead functionalized with a moiety to bind to the binder moiety, and separating the first set of supports from the second and third sets of supports.

In an example of the first aspect and the above examples, the method further includes applying to a dispersion of the second and third sets of supports, a second capture primer complementary to the third region and the first type of sequence, the second capture primer including a binder moiety, applying a magnetic bead functionalized with a moiety to bind to the binder moiety, and separating the second set of supports from the third set of supports.

In another example of the first aspect and the above examples, the method further includes combining the separated second set of supports with the separated first set of supports to form a mixed monoclonal set of supports.

In a further example of the first aspect and the above examples, the method further includes depositing the mixed monoclonal set of supports onto a sequencing substrate.

In an additional example of the first aspect and the above examples, the method further includes sequencing the target nucleic acids disposed on supports of the mixed monoclonal set of supports deposited on the sequencing substrate.

In another example of the first aspect and the above examples, sequencing the target nucleic acids includes applying a sequencing primer complementary to at least the first region of the second segment.

In a further example of the first aspect and the above examples, the sequencing primer is not complementary to the second region of the second segment.

In an additional example of the first aspect and the above examples, the sequencing primer is not complementary to the third region of the second segment.

In another example of the first aspect and the above examples, the method further includes preparing a target nucleic acid of the population by extending a template nucleic acid complementary to a fusion primer. The template nucleic acid includes a first segment at the 5′ end, a second segment at 3′ end, and a third segment between the first and second segments. The second segment includes a first region, and the fusion primer includes a first primer region at 3′ end, a third primer region at the 5′ end, and a second primer region between the first and third primer regions. The third primer region is complementary to the third region of the second segment of the target nucleic acid, and the second primer region is complementary to a sequence of one type of the two or more types of sequences of the second region of the second segment of the target nucleic acid.

In a further example of the first aspect and the above examples, extending includes a single polymerase chain reaction cycle.

In an additional example of the first aspect and the above examples, the third region of the second segment includes between 5 and 50 nucleotides. In an example of the first aspect and the above examples, the third region of the second segment includes between 10 and 30 nucleotides.

In another example of the first aspect and the above examples, the second region of the second segment includes between 5 and 50 nucleotides. In an example of the first aspect and the above examples, the second region of the second segment includes between 10 and 30 nucleotides.

In a further example of the first aspect and the above examples, the first region of the second segment includes between 5 and 50 nucleotides. In an example of the first aspect and the above examples, the first region of the second segment includes between 10 and 30 nucleotides.

In an additional example of the first aspect and the above examples, the second region of the second segment is disposed at the 3′ end of the first region of the second segment.

In another example of the first aspect and the above examples, the third region of the second segment is disposed at the 3′ end of the second region of the second segment.

In a further example of the first aspect and the above examples, amplifying includes performing cycles of polymerase chain reaction.

In an additional example of the first aspect and the above examples, amplifying includes performing isothermal amplification.

In another example of the first aspect and the above examples, each target nucleic acid further includes a barcode segment disposed between the second segment and the third segment.

In a further example of the first aspect and the above examples, each target nucleic acid further includes a key segment disposed between the second segment and the third segment.

In a second aspect of the disclosure, a method for isolating monoclonal supports is provided. The method includes preparing a population of bead supports including first, second, and third sets of bead supports bound to target nucleic acids. Each target nucleic acid includes a first segment at the 5′ end, a second segment at 3′ end, and a third segment disposed between the first and second segments. The second segment includes a first region, a second region, and a third region, wherein the first segment is the same for each target nucleic acid of the population, the first and third regions of the second segment are the same for each target nucleic acid of the population, and the second region of the second segment has a sequence of one type of two or more types of sequences. The first set of bead supports includes a monoclonal population of target nucleic acids having a second region with a sequence of a first type, the second set of bead supports includes a monoclonal population of target nucleic acids having a second region with a sequence of a second type, and the third set of bead supports includes a polyclonal population of target nucleic acids having second regions with sequences of both types. The method further involves adding an enrichment probe complementary to the third region and the second region having the sequence of a second type, the enrichment probe including a capture moiety, adding magnetic beads with moieties that bind to the capture moiety, and separating the first set of bead supports from the second and third sets of bead supports.

In an example of the second aspect and the above examples, the method further includes adding to the second and third sets of bead supports, a second enrichment probe complementary to the third region and the second region having the sequence of a first type, the enrichment probe including a capture moiety, adding magnetic beads with moieties that bind to the capture moiety, and separating the second set of bead supports from the third set of bead supports.

In another example of the second aspect and the above examples, the method further includes combining the first and second sets of bead supports to form a mixed monoclonal set of supports.

In a further example of the second aspect and the above examples, the method further includes depositing the mixed monoclonal set of supports onto a sequencing substrate.

In an additional example of the second aspect and the above examples, the method further includes sequencing the target nucleic acids disposed on supports of the mixed monoclonal set of supports deposited on the sequencing substrate.

In another example of the second aspect and the above examples, sequencing the target nucleic acids includes applying a sequencing primer complementary to at least the first region of the second segment. In an example of the second aspect and the above examples, the sequencing primer is not complementary to the second region of the second segment. In an example of the second aspect and the above examples, the sequencing primer is not complementary to the third region of the second segment.

In a further example of the second aspect and the above examples, the third region of the second segment includes between 5 and 50 nucleotides. In an example of the second aspect and the above examples, the third region of the second segment includes between 10 and 30 nucleotides.

In an additional example of the second aspect and the above examples, the second region of the second segment includes between 5 and 50 nucleotides. In an example of the second aspect and the above examples, the second region of the second segment includes between 10 and 30 nucleotides.

In another example of the second aspect and the above examples, the first region of the second segment includes between 5 and 50 nucleotides. In an example of the second aspect and the above examples, the first region of the second segment includes between 10 and 30 nucleotides.

In a further example of the second aspect and the above examples, the second region of the second segment is disposed at the 3′ end of the first region of the second segment.

In an additional example of the second aspect and the above examples, the third region of the second segment is disposed at the 3′ end of the second region of the second segment.

In another example of the second aspect and the above examples, amplifying includes performing cycles of polymerase chain reaction.

In a further example of the second aspect and the above examples, amplifying includes performing isothermal amplification.

In an additional example of the second aspect and the above examples, amplifying includes performing a two-step isothermal amplification.

In another example of the second aspect and the above examples, each target nucleic acid further includes a barcode segment disposed between the second segment and the third segment.

In a further example of the second aspect and the above examples, each target nucleic acid further includes a key segment disposed between the second segment and the third segment.

In a third aspect of the disclosure, a method of forming target nucleic acids is provided. The method includes preparing a population of target nucleic acids, each target nucleic acid having a first segment at the 5′ end, a second segment at 3′ end, and a third segment between the first segment and the second segment. The first segment has the same sequence for each of the target nucleic acids of the population, and the second segment has the same sequence for each of the target nucleic acids of the population. The method further includes applying a fusion primer, which includes at the 3′ end a region complementary to the second segment of a target nucleic acid, an enrichment barcode region, and an enrichment primer region at the 5′ end. The enrichment barcode region has an enrichment barcode sequence of a type selected from two or more types of enrichment barcode sequences. The method also includes extending the target nucleic acid complementary to the fusion primer.

In an example of the third aspect and the above examples, the method further includes extending the fusion primer complementary to the target nucleic acid.

In another example of the third aspect and the above examples, extending includes a single polymerase chain reaction cycle.

In a further example of the third aspect and the above examples, extending includes isothermal amplification.

In an additional example of the third aspect and the above examples, the third region of the second segment includes between 5 and 50 nucleotides. In an example of the third aspect and the above examples, the third region of the second segment includes between 10 and 30 nucleotides.

In another example of the third aspect and the above examples, the second region of the second segment includes between 5 and 50 nucleotides. In an example of the third aspect and the above examples, the second region of the second segment includes between 10 and 30 nucleotides.

In a further example of the third aspect and the above examples, the first region of the second segment includes between 5 and 50 nucleotides. In an example of the third aspect and the above examples, the first region of the second segment includes between 10 and 30 nucleotides.

In an additional example of the third aspect and the above examples, each target nucleic acid further includes a barcode segment disposed between the second segment and the third segment.

In another example of the third aspect and the above examples, each target nucleic acid further includes a key segment disposed between the second segment and the third segment.

In a fourth aspect of the disclosure, a method of forming target nucleic acids is provided. The method includes preparing a population of target nucleic acids, each target nucleic acid having a first segment at the 5′ end, a second segment at 3′ end, and a third segment between the first segment and the second segment. The first segment has the same sequence for each of the target nucleic acids of the population, and the second segment has the same sequence for each of the target nucleic acids of the population. The method further includes applying a population of fusion primers, each fusion primer including at 3′ end a region complementary to the second segment of a target nucleic acid, an enrichment barcode region, and an enrichment primer region at the 5′ end. For a first set of fusion primers, the enrichment barcode region has an enrichment barcode sequence of a first type selected from two or more types of enrichment barcode sequences. For a second set of fusion primers, the enrichment barcode region has an enrichment barcode sequence of a second type selected from the two or more types of enrichment barcode sequences. The method also includes extending the target nucleic acid complementary to the fusion primer.

In an example of the fourth aspect and the above examples, the method further includes extending the fusion primer complementary to the target nucleic acid.

In another example of the fourth aspect and the above examples, extending includes a single polymerase chain reaction cycle.

In a further example of the fourth aspect and the above examples, extending includes isothermal amplification.

In an additional example of the fourth aspect and the above examples, the third region of the second segment includes between 5 and 50 nucleotides. In an example of the fourth aspect and the above examples, the third region of the second segment includes between 10 and 30 nucleotides.

In another example of the fourth aspect and the above examples, the second region of the second segment includes between 5 and 50 nucleotides. In an example of the fourth aspect and the above examples, the second region of the second segment includes between 10 and 30 nucleotides.

In a further example of the fourth aspect and the above examples, the first region of the second segment includes between 5 and 50 nucleotides. In an example of the fourth aspect and the above examples, the first region of the second segment includes between 10 and 30 nucleotides.

In an additional example of the fourth aspect and the above examples, each target nucleic acid further includes a barcode segment disposed between the second segment and the third segment.

In another example of the fourth aspect and the above examples, each target nucleic acid further includes a key segment disposed between the second segment and the third segment.

In a fifth aspect of the disclosure, a method of sequencing a population of target nucleic acids is provided. The method includes applying sequencing primers to a population of target nucleic acids, each target nucleic acid including a first segment at the 5′ end, a second segment at 3′ end, and a third segment disposed between the first and second segments. The second segment includes a first region, a second region, and a third region. The first segment is the same for each target nucleic acid of the population, the first and third regions of the second segment are the same for each target nucleic acid of the population, and the second region of the second segment has a sequence of one type of two or more types of sequences. The sequencing primers are complementary to the first region of the second segment of each target nucleic acid. The method further includes incorporating at least one nucleotide to extend a sequencing primer and detecting the incorporation.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

1. A method for enriching clonal populations, the method comprising:
providing a population of target nucleic acids, each target nucleic acid of the population of target nucleic acids including a first segment at the 5′ end, a second segment at 3′ end, and a third segment disposed between the first and second segments, the second segment including a first region, a second region, and a third region, wherein the first segment is the same for each target nucleic acid of the population of nucleic acids, the first and third regions of the second segment are the same for each target nucleic acid of the population of nucleic acids, the second region of the second segment having a sequence of one type of two or more types of sequences;
exposing the population of target nucleic acids to a plurality of supports, each support of the plurality of supports having a set of bound oligonucleotides the same as or complementary to the first segment, target nucleic acids of the population of target nucleic acids or complements thereto bind to supports of the plurality of supports;
amplifying the bound target nucleic acids in the presence of a primer at least complementary to the third region of the second segment to form supports including a plurality of copies of the target nucleic acids of the population of target nucleic acids, wherein a first set of supports includes monoclonal copies of a target nucleic acid of the population of target nucleic acids having a second region having a sequence of a first type of sequence of the two or more types of sequences, a second set of supports includes monoclonal copies of a target nucleic acid of the population of target nucleic acids having a second region having a sequence of a second type of sequence of the two or more types of sequences, and a third set of supports includes a polyclonal mixed set of copies of target nucleic acids having second regions with sequences of two or more types of the two or more types of sequences;
applying to the supports a capture primer have a sequence complementary to the third region and to the second type of the two or more types of sequences, the capture primer including a binder moiety;
applying a magnetic bead functionalize with a moiety to bind to the binder moiety; and
separating the first set of supports from the second and third set of supports.

2. The method of claim 1, further comprising:
applying to a dispersion of the second and third sets of supports, a second capture primer complementary to the third region and the first type of sequence of the two or more types of sequences and including a binder moiety;
applying a magnetic bead functionalize with a moiety to bind to the binder moiety; and
separating the second set of supports from the third set of supports.

3. The method of claim 2, further comprising combining the separated second set of supports with the separated first set of supports to form a mixed monoclonal set of supports.

4. The method of claim 3, further comprising depositing the mixed monoclonal set of supports on to a sequencing substrate.

5. The method of claim 4, further comprising sequencing the target nucleic acids disposed on supports of the mixed monoclonal set of supports disposed on the sequencing substrate.

6. The method of claim 5, wherein sequencing the target nucleic acids includes applying a sequencing primer complementary to at least the first region of the second segment.

7. The method of claim 6, wherein the sequencing primer is not complementary to the second region of the second segment.

8. The method of claim 6, wherein the sequencing primer is not complementary to the third region of the second segment.

9. The method of claim 1, further comprising preparing a target nucleic acid of the population of target nucleic acids by extending a template nucleic acid complementary to a fusion primer, the template nucleic acid including a first segment at the 5′ end, a second segment at 3′ end, and a third segment disposed between the first and second segments, the second segment including a first region, the fusion primer including a first primer region at 3′ end of the fusion primer, a third primer region at the 5′ end of the fusion primer, and a second primer region disposed between the first primer region and the third primer region, the third primer region complementary to the third region of the second segment of the target nucleic acid, the second primer region complementary to a sequence of one type of the two or more types of sequences of the second region of the second segment of the target nucleic acid.

10. The method of claim 9, wherein extending include a single polymerase chain reaction cycle.

11. The method of claim 1, wherein the third region of the second segment includes between 5 and 50 nucleotides.

12. The method of claim 11, wherein the third region of the second segment includes between 10 and 30 nucleotides.

13. The method of claim 1, wherein the second region of the second segment includes between 5 and 50 nucleotides.

14. (canceled)

15. The method of claim 1, wherein the first region of the second segment includes between 5 and 50 nucleotides.

16. (canceled)

17. The method of claim 1, wherein the second region of the second segment is disposed at the 3′ end of the first region of the second segment.

18. The method of claim 1, wherein the third region of the second segment is disposed at the 3′ end of the second region of the second segment.

19. The method of claim 1, wherein amplifying includes performing cycles of polymerase chain reaction.

20. The method of claim 1, wherein amplifying includes performing isothermal amplification.

21. The method of claim 1, wherein each target nucleic acid further includes a barcode segment disposed between the second segment and the third segment.

22. The method of claim 1, wherein each target nucleic acid further includes a key segment disposed between the second segment and the third segment.

23.-68. (canceled)
